(19) United States
(12) Patent Application Publication
(10) Pub. No.: US 2026/0015663 A1
(43) Pub. Date: Jan. 15, 2026

(54) MILK-DERIVED MIRNAS AS INDICATORS OF STRESS IN BOVINE

(71) Applicant: SOCIÉTÉ DES PRODUITS NESTLÉ S.A., Vevey (CH)

(72) Inventors: Maxim BOBKOV, Epalinges (CH); Cristina LECCHI, Stezzano (Bg) (IT); Fabrizio CECILIANI, Pavia (IT); Pasquale DE PALO, Ruvo di Puglia (BA) (IT); Aristide MAGGIOLINO, Bisceglie (BT) (IT); Elodie Audrey SOUSSAN, Epalinges (CH); Frederic RAYMOND, Châtel-St-Denis (CH)

(21) Appl. No.: 19/140,853

(22) PCT Filed: Dec. 15, 2023
(86) PCT No.: PCT/EP2023/086206

§ 371 Date: Jun. 18, 2025

(30) Foreign Application Priority Data
Dec. 20, 2022 (EP) 22215067.4

(51) Int. Cl. C12Q 1/6876

(52) U.S. Cl. CPC C12Q 1/6876; C12Q 2600/124; C12Q 2600/158; C12Q 2600/178

(57) ABSTRACT

The present invention relates miRNA biomarkers from milk. e.g. exosome-derived microRNA from milk, as indicators of welfare in bovines. In particular, the present invention relates to the identification of heat stress in bovines allowing to propose management actions to improve productive animal welfare at the individual or the herd levels.

### TECHNICAL FIELD OF THE INVENTION

This present invention relates to the use of milk-derived micro-RNAs of dairy cattle milk as indicators of animal welfare, such as indicators of stress such as heat stress.

### BACKGROUND OF THE INVENTION

International regulatory framework proposes observations of changes in animal health status and behaviour to define if the animal(s) are stressed and to propose improvements in animal welfare management. However, a visual observation alone is subjective and does not consider specific physiological markers of stress.

Currently, different indicators are used to evaluate if an animal has or is experiencing stress conditions. These include,
observations in changes in animal health status and behaviour, which is currently considered the reference tool to monitor and detect early warning signs in animal health and welfare deterioration;
changes in blood parameters, such as glycemia, blood cell counts, levels of hormones and metabolites. However, these parameters are highly variable across different animal breeds, age, stage of the life. Moreover, the level of hormones such as cortisol passing in milk is too variable, and as such classical biomarkers are not suitable stress specific indicators.

By contrast, the expression of miRNA is specific to cells and tissues and well-regulated.

Ioannidis et al. discloses associations of plasma microRNA expression with age, genetic background and functional traits in dairy cattle (SCIENTIFIC REPORTS|(2018) 8:12955).

Miretti et al. discloses microRNAs as biomarkers for animal health and welfare in Livestock (Front. Vet. Sci. 7:578193).

Billa et al. discloses that nutrigenomic analyses reveal miRNAs and mRNAs affected by feed restriction in the mammary gland of midlactation dairy cows (PLoS ONE 16 (4): e0248680).

Li et al. (BMC Genomics (2018) 19:975) discloses a characterization of miRNA profiles in the mammary tissue of dairy cattle in response to heat stress.

Hence, an improved method to non-invasively determine animal welfare would be advantageous, and in particular a more efficient and/or reliable method to determine animal stress, such as heat stress would be advantageous. The present invention addresses this need.

### SUMMARY OF THE INVENTION

The present invention relates to miRNA biomarkers from milk, e.g. exosome-derived microRNA from milk, as indicators of welfare or a welfare parameter in cattle. In particular, the present invention relates to the identification of stress, particularly heat stress in cattle, which in turn means actions can be taken to manage and improve productive animal welfare at the individual or the herd level.

Thus, an object of the present invention relates to the provision of miRNA biomarkers, which can be indicative of welfare parameters in cattle, preferably dairy cattle.

In particular, it is an object of the present invention to provide miRNA biomarkers from milk-derived exosomes, which can indicate heat stress in (dairy) cattle and which can be used across different breeds of (dairy) cattle.

Example 1 shows the characterization of physiological parameters, as well as oxidative and inflammatory markers in biologicals samples from cows of two breeds in thermal comfort conditions and under heat stress. This example shows that heat stress affects the welfare of cattle and influences production parameters.

Example 2 is an analysis of the miRNA profile of milk-derived exosomes using Next Generation Sequencing (NGS) in milk from cows of two different breeds in thermal comfort conditions and heat stress.

Example 3 shows the identification of miRNAs that expressed differently in milk from cows of two breeds in heat stress.

Example 4 shows the identification of miRNA expressed similarly in milk from cows of two breeds in heat stress.

Thus, one aspect of the invention relates to a method of determining at least one welfare parameter of at least one head of cattle, the method comprising
i. determining in a milk sample the level of at least one miRNA that regulates (at least) the cell cycle, wherein the miRNA is selected from SEQ ID NO: 94, 123, 93, 84, 81, 134, 85, 74, 83, 113, 1, 132, 112, and 111 or a functional variant thereof, wherein the functional variant has at least 60% overall sequence identity to SEQ ID NO: 94, 123, 93, 84, 81, 134, 85, 74, 83, 113, 1, 132, 112, and 111 respectively; and
ii. comparing the level of the at least one miRNA to a reference level, wherein if said one or more miRNA selected from SEQ ID NO: 94, 123, 93, 84, 85, 74, 83, 113, 1, 132, 112, and 111 is above said reference level at least one welfare parameter of the at least one head of cattle has been or is compromised or if said one or more miRNA selected from SEQ ID NO: 81 and 134 is below said reference level at least one welfare parameter of the at least one head of cattle has been or is compromised.

In another aspect of the invention, there is provided a method of improving at least one welfare parameter of at least one head of cattle, the method comprising
i. determining in a first milk sample the level of at least one miRNA that regulates the cell cycle, wherein the miRNA is selected from SEQ ID NO: 94, 123, 93, 84, 81, 134, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111 respectively;
ii. comparing the level of the at least one miRNA to a reference level, wherein if said one or more miRNA selected from SEQ ID NO: 94, 123, 93, 84, 85, 74, 83, 113, 1, 132, 112, and 111 is above said one or more reference level at least one welfare parameter of the at least one head of cattle has been compromised or if said one or more miRNA selected from SEQ ID NO: 81 and 134 is below said one or more reference level at least one welfare parameter of the at least one head of cattle has been compromised;
iii. improving the welfare of the at least one head of cattle;
iv. determining in a second milk sample the level of at least one miRNA selected from SEQ ID NO: 94, 123, 93, 84, 81, 134, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111 respectively; and
v. comparing the level of the at least one miRNA in the first milk sample to the level in the second milk sample, wherein if the level of SEQ ID NO: 94, 123, 93, 84, 85, 74, 83, 113, 1, 132, 112, and 111 or 55 or a functional variant thereof wherein the functional variant has at least 60% sequence identity to SEQ ID NO: 94, 123, 93, 84, 85, 74, 83, 113, 1, 132, 112, and 111 respectively in the second milk sample is lower than the level in the first milk sample or lower than the level in the reference sample the welfare of the head of cattle has improved; or if the level of SEQ ID NO: 81 or 134 in the second milk sample is higher than the level in the first milk sample or higher than the reference sample the welfare of the head of cattle has improved.

In one embodiment, improving the welfare of the at least one head of cattle comprises one or more of changing heat exposure, improving access to additional water, changing the feed, reducing animal density and the provision of cooling systems.

In a further aspect of the invention, there is provided a method of improving at least one of milk yield, milk quality, meat yield or meat quality of at least one head of cattle, the method comprising
i. determining in a first milk sample the level of at least one miRNA involved in regulating the cell cycle, wherein the miRNA is selected from SEQ ID NO: 94, 123, 93, 84, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111;
ii. comparing the level of the at least one miRNA to a reference level, wherein if said one or more miRNA selected from SEQ ID NO: 94, 123, 93, 84, 85, 74, 83, 113, 1, 132, 112, and 111 is above said one or more reference level at least one welfare parameter of the at least one head of cattle has been or is compromised or if said one or more miRNA selected from SEQ ID NO: 81 and 134 is below said one or more reference level at least one welfare parameter of the at least one head of cattle has been compromised; and
iii. improving the welfare of the at least one head of cattle; wherein improving the welfare of the at least one head of cattle improves at least one of milk yield, milk quality, meat yield and meat quality.

In another aspect of the invention, there is provided a method of determining at least one welfare parameter of at least one head of cattle, the method comprising determining the level of at least one miRNA, wherein the miRNA is selected from SEQ ID NO: 94, 123, 93, 84, 2, 81, 134, 85, 52, 74, 83, 6, 113, 70, 1, 69, 132, 120, 48, 112, 111, 31, 87 and 55 or a functional variant thereof, wherein said functional variant has at least 60% overall sequence identity to SEQ ID NO: 94, 123, 93, 84, 2, 81, 134, 85, 52, 74, 83, 6, 113, 70, 1, 69, 132, 120, 48, 112, 111, 31, 87 and 55 respectively; wherein when the level of SEQ ID NO: 94, 123, 93, 84, 85, 52, 74, 83, 6, 113, 70, 1, 69, 132, 120, 48, 112, 111, 31, 87 and 55 or a functional variant thereof is increased by 1 log 2FC (1 log fold change) or more compared to a reference level or wherein the level of SEQ ID NO: 2, 81 or 134 is decreased by −1 log 2FC or more compared to a reference level, at least one welfare parameter of the at least one head of cattle has been or is not compromised.

In an embodiment, if the level of SEQ ID NO: 94 or a functional variant thereof is above said one or more reference level at least one welfare parameter of the at least one head of cattle has been compromised.

In one embodiment, the welfare parameter of at least one head of cattle may be or is comprised by at least one type of stress, examples of which include but are not limited to heat stress, social stress, metabolic stress, disease stress or combinations thereof.

In one embodiment, the cattle is dairy cattle, preferably bovine. In another embodiment, the breed of cattle is selected from Holstein and/or Brown Swiss.

In one embodiment, the milk sample is from a herd of cattle.

The method may comprise determining the level of all of SEQ ID NO: 94, 123, 93, 84, 85, 74, 83, 113, 1, 132, 112 and 111 or a functional variant thereof wherein the functional variant has at least 60% sequence identity to SEQ ID NO's: 94, 123, 93, 84, 81, 134, 85, 74, 83, 113, 1, 132, 112 and 111 respectively.

The reference level may be the level of the one or more miRNA in a milk sample from at least one head of cattle where the welfare of the head of cattle is not or has not been compromised or wherein the reference level is an average level from cattle where the welfare of the cattle is not or has not been compromised.

The method may further comprise determining in a milk sample from said head of cattle, the level of a physiological parameter, wherein the physiological parameter may be selected from one or more of heat shock protein 70, a serum protein profile, blood urea nitrogen, blood uric acid, serum chloride, serum phosphorous, serum triglyceride and serum magnesium.

In one embodiment the method comprises determining the level of heat shock protein 70 in a milk sample from said head of cattle, comparing said level to corresponding reference level of heat shock protein 70 (a reference level is defined herein), wherein where the level of heat shock protein 70 is increased compared to the reference level it is indicative that at least one welfare parameter (i.e. the welfare) of the at least one head of cattle has been compromised.

In one embodiment the method comprises determining the serum protein profile in a milk sample from said head of cattle, comparing said level to corresponding reference serum protein profile (a reference level is defined herein), wherein if total proteins are increased compared to the reference level it is indicative that at least one welfare parameter (i.e. the welfare) of the at least one head of cattle has been compromised.

In one embodiment the method comprises determining the level of blood urea nitrogen in a milk sample from said head of cattle, comparing said level to corresponding reference level of blood urea nitrogen (a reference level is defined herein), wherein where the level of blood urea nitrogen is increased compared to the reference level it is indicative that at least one welfare parameter (i.e. the welfare) of the at least one head of cattle has been compromised.

In one embodiment the method comprises determining the level of the blood uric acid in a milk sample from said head of cattle, comparing said level to corresponding reference level of the blood uric acid (a reference level is defined herein), wherein where the level of the blood uric acid is decreased compared to the reference level it is indicative that at least one welfare parameter (i.e. the welfare) of the at least one head of cattle has been compromised.

In one embodiment the method comprises determining the level of serum chloride in a milk sample from said head of cattle, comparing said level to corresponding reference level of serum chloride (a reference level is defined herein), wherein where the level of serum chloride is increased compared to the reference level it is indicative that at least one welfare parameter (i.e. the welfare) of the at least one head of cattle has been compromised.

In one embodiment the method comprises determining the level of serum phosphorous in a milk sample from said head of cattle, comparing said level to corresponding reference level of serum phosphorous (a reference level is defined herein), wherein where the level of serum phosphorous is increased compared to the reference level it is indicative that at least one welfare parameter (i.e. the welfare) of the at least one head of cattle has been compromised.

In one embodiment the method comprises determining the level of serum magnesium in a milk sample from said head of cattle, comparing said level to corresponding reference level of serum magnesium (a reference level is defined herein), wherein where the level of serum magnesium is decreased compared to the reference level it is indicative that at least one welfare parameter (i.e. the welfare) of the at least one head of cattle has been compromised.

In one embodiment the method comprises determining the level of serum triglyceride in a milk sample from said head of cattle, comparing said level to corresponding reference level of serum triglyceride (a reference level is defined herein), wherein where the level of triglyceride is decreased compared to the reference level it is indicative that at least one welfare parameter (i.e. the welfare) of the at least one head of cattle has been compromised.

In another aspect of the invention, there is provided a device or system adapted to determine if at least one welfare parameter of at least one head of cattle has been or is compromised, the device comprising:
a unit able to determine the level of one or more miRNAs described herein;
a processor configured with a reference table, said reference table corresponding to reference levels of one or more of the miRNA according to Table 4;
wherein the device or system is adapted to
receive the milk sample;
determine the levels of the one or more miRNAs;
compare the determined levels of the one or more miRNAs to the reference table; and
determine if the welfare of the at least one head of cattle has been or is compromised.

In another aspect of the invention, there is provided a computer implemented method of determining if the welfare of at least one head of cattle has been compromised, the method comprising:
providing levels of one or more miRNAs from a cattle milk sample selected from Table 4;
providing a mathematical model comprising reference levels of one or more corresponding miRNAs selected from Table 4;
determining if the provided levels deviates significantly from the reference levels, using the mathematical model; and
providing to a system or a user a determination of whether the welfare of the at least one head of cattle has been or is compromised.

In another aspect of the invention, there is provided a kit for evaluating at least one welfare parameter of at least one head of cattle comprising the device of the invention and instructions for use.

In a further embodiment of any of the above described methods, uses and devices or systems, the miRNA is alternatively that defined in SEQ ID NO: 2 or a functional variant thereof, wherein the functional variant has at least 60% overall sequence identity to SEQ ID NO: 2.

In a further embodiment of any of the above described methods, uses and devices or systems, the miRNA is alternatively that defined in SEQ ID NO: 52 or a functional variant thereof, wherein the functional variant has at least 60% overall sequence identity to SEQ ID NO: 52.

In a further embodiment of any of the above described methods, uses and devices or systems, the miRNA is alternatively that defined in SEQ ID NO: 6 or a functional variant thereof, wherein the functional variant has at least 60% overall sequence identity to SEQ ID NO: 6.

In a further embodiment of any of the above described methods, uses and devices or systems, the miRNA is alternatively that defined in SEQ ID NO: 70 or a functional variant thereof, wherein the functional variant has at least 60% overall sequence identity to SEQ ID NO: 70.

In a further embodiment of any of the above described methods, uses and devices or systems, the miRNA is alternatively that defined in SEQ ID NO: 69 or a functional variant thereof, wherein the functional variant has at least 60% overall sequence identity to SEQ ID NO: 69.

In a further embodiment of any of the above described methods, uses and devices or systems, the miRNA is alternatively that defined in SEQ ID NO: 120 or a functional variant thereof, wherein the functional variant has at least 60% overall sequence identity to SEQ ID NO: 120.

In a further embodiment of any of the above described methods, uses and devices or systems, the miRNA is alternatively that defined in SEQ ID NO: 48 or a functional variant thereof, wherein the functional variant has at least 60% overall sequence identity to SEQ ID NO: 48.

In a further embodiment of any of the above described methods, uses and devices or systems, the miRNA is alternatively that defined in SEQ ID NO: 31 or a functional variant thereof, wherein the functional variant has at least 60% overall sequence identity to SEQ ID NO: 31.

In a further embodiment of any of the above described methods, uses and devices or systems, the miRNA is alternatively that defined in SEQ ID NO: 87 or a functional variant thereof, wherein the functional variant has at least 60% overall sequence identity to SEQ ID NO: 87.

In a further embodiment of any of the above described methods, uses and devices or systems, the miRNA is alternatively that defined in SEQ ID NO: 55 or a functional variant thereof, wherein the functional variant has at least 60% overall sequence identity to SEQ ID NO: 55.

The present invention will now be described in more detail in the following passages.

### BRIEF DESCRIPTION OF THE FIGURES

FIG. 1 shows a schematic of the experimental design used.

### DETAILED DESCRIPTION OF THE INVENTION

### Definitions

Prior to discussing the present invention in further details, the following terms and conventions will first be defined:

### Head of Cattle

At least one head of cattle refers to one animal of unspecified age or gender.

### Dairy Cattle

Dairy cattle (also called dairy cows) are cattle bred for the ability to produce large quantities of milk, from which dairy products are made. Dairy cattle generally are of the species Bos taurus.

### Functional Variant

The term ‘variant’ or ‘functional variant’ refers to a miRNA sequence where the nucleotides of the miRNA are substantially identical to one of the recited sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the recited sequences, such as SEQ ID NOs 94, 2, 81, 134, 85, 52, 74, 83, 6, 113, 70, 1, 69, 132, 123, 120, 48, 112, 84, 111, 31, 87, 55 and 93, preferably over the full length of the sequence. In one embodiment, sequence identity is at least 90%. In another embodiment, sequence identity is 100%. Sequence identity can be determined by any one known sequence alignment program in the art. For the avoidance of doubt, a functional variant performs the same function as the non-variant sequence. For example, a functional variant of an miRNA that regulates the cell cycle also regulates the cell cycle in the same way as the non-variant sequence.

### Increase

In the context of the present invention an increase is an increase in the level of a miRNA by up to or more than 1%, 5%, 10%, 20%, 30%, 40%, 50%, 60%, 70%, 80%, 90% or 100% compared to the reference level. Alternatively, an increase as used herein is an increase by up to or at least 1 log₂FC, 1.5 log₂FC, 2 log₂FC, 3 log₂FC, 4 log₂FC, 5 log₂FC, 6 log₂FC, 7 log₂FC 8 log₂FC, 9 log₂FC, 10 log₂FC or more compared to the reference level.

### Decrease

In the context of the present invention a decrease is a decrease in the level of an miRNA by up to or at least 1%, 2%, 3%, 4%, 5%, 6%, 7%, 8%, 9%, 10%, 20%, 30%, 40%, 50%, 60%, 70%, 80%, 90 or 95% compared to the reference level. Alternatively, as used herein a decrease is a decrease by up to or at least-1 log₂FC, −1.5 log₂FC, −2 log₂FC, −3 log₂FC, −4 log₂FC, −5 log₂FC, −6 log₂FC, −7 log₂FC, −8 log₂FC, −9 log₂FC, −10 log₂FC or more compared to the reference level.

### Log 2FC

Log 2FC stands for log 2 fold change, which is a statistical measure used to quantify the change in expression of a nucleic acid between two conditions. The fold change is simply the ratio of the expression levels of the nucleic acid in the two conditions and the log 2 transformation is used to normalise the data for interpretation. A log 2FC of 1 indicates that the expression of a miNA has doubled, while a log 2FC of 2 indicates that the expression of a miRNA has quadrupled. The log 2FC is used to identify miRNAs that are differentially expressed under different conditions.

### Welfare

Welfare is a broad term, which includes the many elements that contribute to an animal's quality of life, including those referred to in the ‘five freedoms’:
I. freedom from hunger, thirst and malnutrition;
II. freedom from fear and distress;
III. freedom from physical and thermal (heat) discomfort;
IV. freedom from pain, injury and disease; and
V. freedom to express normal patterns of behaviour.

If one of those five freedoms (“welfare parameters”) are not fulfilled, a freedom or welfare parameter is or has been considered to be compromised. For example, if an animal is exposed to heat stress, said parameter is considered to be compromised. Thus, in the present context, the term “compromised welfare parameter” refers to one of these five freedoms not being fulfilled for the animal or herd of animals. By “has been comprised” may mean that the welfare was comprised in an animal. The phrasing “at least one welfare parameter” and “welfare” may be used interchangeably.

When a welfare parameter is compromised, it means that the animal is not receiving the level of welfare they should be. Welfare parameters include but are not limited to nutritional welfare, physical welfare, behavioural welfare and environmental welfare. Action can then be taken to improve the welfare of the animal or animals.

Animal welfare means the physical and mental state of an animal in relation to the conditions in which it lives and dies. An animal experiences good welfare if the animal is healthy, comfortable, well nourished, safe, is not suffering from unpleasant states such as pain, fear and distress (such as heat stress), and is able to express behaviours that are important for its physical and mental state.

Some measures of animal welfare involve assessing the degree of impaired functioning associated with injury, disease and malnutrition. Other measures provide information on animals' needs and affective states such as hunger, pain and fear, often by measuring the strength of animals' preferences, motivations and aversions. Others assess the physiological, behavioural and immunological changes or effects that animals show in response to various challenges.

Such measures can lead to criteria and indicators that help to evaluate how different methods of managing animals influence their welfare.

Many aspects of the environment can impact the welfare of animals. The risk of heat stress for cattle is influenced by environmental factors including air temperature, relative humidity, wind speed, animal density (area and volume available per animal), shade availability, animal factors including breed, age, body condition, metabolic rate and stage of lactation, and coat colour and density.

In a preferred embodiment, compromised welfare is determined as cattle being exposed to heat stress. Heat stress may be defined by the “temperature-humidity index (THI)”.

### Temperature-Humidity Index (THI)

The temperature-humidity index (THI) is a single value representing the combined effects of air temperature and humidity associated with the level of thermal stress. This index has been developed as a weather safety index to monitor and reduce heat-stress-related losses. Different animal species and humans have different sensitivities to ambient temperature and the amount of moisture in the air. During hot and humid weather the natural capability of cattle to dissipate heat load by sweating and panting is compromised and heat stress occurs. At THI values of 75 to 78, the animal organism is under heat stress, but the mechanisms of thermoregulation still manage to cope, while at THI over 78 it is assumed that the stress is so high that it is impossible to maintain the thermoregulatory mechanisms or normal body temperature.

Thus, in an embodiment, heat stress is defined as the cattle has been exposed to a THI of 75 or above, preferably 76 or above, such as in the range 75-78 or 76-78.

MicroRNA (miRNA)

MicroRNA (miRNA) are small, single-stranded, non-coding RNA molecules containing around 21 to 23 nucleotides.

The level of any given miRNA can be measured using techniques known in the art, such techniques include but are not limited to northern blot, microarray assays, RT-PCR, RNA-SEQ and miRNA-SEQ. These are standard assays in the art.

### Exosomes

Exosomes are membrane-bound extracellular vesicles (EVs) that are produced in the endosomal compartment of most eukaryotic cells. In the present context the terms “exosome”, “extracellular vesicle” and “EV” are used interchangeably.

### Reference Level

In the context of the present invention, the term “reference level” relates to a standard in relation to a quantity, which other values or characteristics can be compared to.

Reference levels can be selected in different ways, known to the person skilled in the art. In an embodiment, said reference level is the level of the one or more miRNA's in a milk sample from a cattle having normal welfare or an average level from several cattle having normal welfare. Preferably from dairy cattle.

In another embodiment, said reference level is the level of the one or more miRNA's in a milk sample from a (dairy) cattle, which is not or has not been exposed to heat stress or an average level from several (dairy) cattle not exposed to heat stress.

In one embodiment of the present invention, it is possible to determine a reference level by investigating the abundance of one or more of the biomarkers according to the invention in milk samples from (dairy) cattle, which are considered to have normal welfare (i.e. not subject to one or more of the forms of stress discussed herein). By applying different statistical means, such as multivariate analysis, one or more reference levels can be calculated.

Based on these results a cut-off may be obtained that shows the relationship between the level(s) detected and (dairy) cattle considered at risk of not having normal welfare. The cut-off can thereby be used to determine the amount of the one or more biomarkers, which corresponds to, for instance, an increased risk of the cattle not having normal welfare.

The cut-off level could be established using a number of methods, including: multivariate statistical tests (such as partial least squares discriminant analysis (PLS-DA), random forest, support vector machine, etc.), percentiles, mean plus or minus standard deviation(s); median value; fold changes.

The multivariate discriminant analysis and other risk assessments can be performed on the free or commercially available computer statistical packages (SAS, SPSS, Matlab, R, etc.) or other statistical software packages or screening software known to those skilled in the art.

As obvious to one skilled in the art, in any of the embodiments discussed above, changing the risk cut-off level could change the results of the discriminant analysis for each sample tested.

Statistics enables evaluation of the significance of each biomarker level. Commonly used statistical tests applied to a data set include t-test, f-test or even more advanced tests and methods of comparing data. Using such a test or method enables the determination of whether two or more samples are significantly different or not.

The significance may be determined by the standard statistical methodology known by the person skilled in the art.

The chosen reference level may be changed depending on the specific sample for which the test is applied.

The chosen reference level may be changed if desired to give a different specificity or sensitivity as known in the art. Sensitivity and specificity are widely used statistics to describe and quantify how good and reliable a biomarker or a diagnostic test is. Sensitivity evaluates how good a biomarker or a diagnostic test is at detecting a disease or state of health, while specificity estimates how likely the tested cattle or group of cattle (i.e. control, cattle having normal welfare) can be correctly identified as having normal welfare.

Several terms are used along with the description of sensitivity and specificity; true positives (TP), true negatives (TN), false negatives (FN) and false positives (FP). If a compromised welfare is proven to be present in cattle with compromised welfare, the result of the test is considered to be TP. If compromised welfare is not present in the cattle (i.e. control, without disease), and the test confirms the absence of compromised welfare, the test result is TN. If the test indicates the presence of compromised welfare in cattle with no such compromised welfare, the test result is FP. Finally, if the test indicates no presence of compromised welfare in cattle with compromised welfare, the test result is FN.

### Sensitivity

Sensitivity=TP/(TP+FN)=number of true positive assessments/number of all samples from cattle with compromised welfare.

As used herein the sensitivity refers to the measures of the proportion of actual positives which are correctly identified as such—in analogy with a diagnostic test, i.e. the percentage of cattle having welfare below normal who are identified as having welfare below normal.

### Specificity

Specificity=TN/(TN+FP)=number of true negative assessments/number of all samples from controls.

As used herein the specificity refers to measures of the proportion of negatives, which are correctly identified—i.e. the percentage of cattle having welfare at a normal level who are identified as having welfare at a normal level. The relationship between both sensitivity and specificity can be assessed by the ROC curve. This graphical representation helps to decide the optimal model through determining the best threshold or cut-off for a test or a biomarker candidate.

As will be generally understood by those skilled in the art, methods for screening are processes of decision-making and therefore the chosen specificity and sensitivity depend on what is considered to be the optimal outcome by a given animal handler/farmer/personnel or buyer of the milk from the cattle or dairy products in question.

It would be obvious for a person skilled in the art that it may be advantageous to select a higher sensitivity at the expense of lower specificity in most cases, to identify as many patients with disease as possible.

In a preferred embodiment, the invention relates to a method with a high specificity, such as at least 70%, such as at least 80%, such as at least 90%, such as at least 95%, such as 100%.

In another preferred embodiment, the invention relates to a method with a high sensitivity, such as at least 80%, such as at least 90%, such as 100%.

In a preferred embodiment, compromised welfare is determined as cattle being exposed to heat stress.

### Method for Evaluating a Welfare Parameter of Cattle

As also outlined above, the present invention relates to miRNA biomarkers obtained by a non-invasive technique, such as extracellular vesicle-/exosome-derived microRNAs from a milk sample, as indicators of whether a welfare parameter has been compromised, such as the animal having been exposed to heat stress. Thus, an aspect of the invention relates to a method for evaluating at least one welfare parameter of cattle, preferably bovine, the method comprising:
determining in a milk sample from said cattle, the level of one or more miRNAs selected from the group consisting of SEQ ID NO's: 94, 2, 81, 134, 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 2, 81, 134, 85, 52, 74, 83, 6, 113, 70, 1, 69, 132, 120, 48, 112, 111, 31, 87 and 55;
comparing said one or more levels to one or more corresponding reference levels;
   wherein if said one or more miRNA levels of SEQ ID NO's: 94 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 85, 52, 74, 83, 6, 113, 70, 1, 69, 132, 120, 48, 112, 111, 31, 87 and 55 are above said one or more reference levels, it is indicative of that at least one welfare parameter of the cattle is compromised; and
   wherein if said one or more miRNA levels of SEQ ID NO's: 94 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 85, 52, 74, 83, 6, 113, 70, 1, 69, 132, 120, 48, 112, 111, 31, 87 and 55 are equal to or below said one or more reference levels, it is indicative of that the at least one welfare parameter of the cattle is not compromised;
   wherein if said one or more miRNA levels of SEQ ID NO's: 2, 81, or 134 or a functional variant wherein the functional variant has at least 60% overall sequence identity to SEQ ID NO: 2, 81, or 134, are equal to or above said one or more reference levels, it is indicative of that the at least one welfare parameter of the cattle is not compromised; and
   wherein if said one or more miRNA levels of SEQ ID NO's: 2, 81 or 134 or a functional variant wherein the functional variant has at least 60% overall sequence identity to SEQ ID NO: 2, 81 or 134, are below said one or more reference levels, it is indicative of that the at least one welfare parameter of the cattle is compromised.

### Welfare

Different welfare parameters may be evaluated using the method of the invention. Thus, in an embodiment, the at least one compromised welfare parameter is caused by a parameter selected from the group consisting of heat stress, social stress, metabolic stress, disease stress, and combinations thereof, preferably the parameter is heat stress. In e.g. examples 2-4, miRNA biomarkers in relation to heat stress have been identified.

Thus, in a preferred embodiment, the compromised welfare parameter is caused by heat stress.

### Cattle

Different kind of cattle may be evaluated using the method of the invention. Hence, in an embodiment, the cattle is dairy cattle. In a related embodiment, the cattle is bovine, such as Bos taurus, more preferably bovine dairy cattle.

The method of the invention may be used on a single cattle or group or herd of cattle. Thus, in an embodiment, said cattle is an individual cattle (a “head of cattle” as referred to herein) or a herd of cattle, preferably a herd of cattle.

In a related embodiment, said milk sample is from a single cattle or a mixed milk sample from a herd of cattle.

As also outlined in the example section, the Applicant has identified a number of biomarkers that can be used irrespective of the breed. However, in an embodiment the cattle is of the breed Brown Swiss and/or Holstein.

### Milk Sample

Different types of milk samples can be used in the method of the invention. Thus, in an embodiment, the milk sample is in the form of raw milk, skimmed milk, milk powder, or a food product or food ingredient comprising milk, dairy-containing products, such as an infant formula, cheese or yoghurt or being derived from such food product or food ingredient.

If the milk sample is a mixed sample from several individual milk samples, it can also be considered “bulk milk”.

In an embodiment, the level of miRNA, is the level of miRNA in milk-derived exosomes. In examples 2-4, the level of miRNA has been determined from milk-derived exosomes.

In a preferred embodiment the miRNA is derived from a milk-derived exosome that expresse CD9 and/or TSG101.

### Biomarkers

As shown in example 4, different individual biomarkers have been identified, which can be used cross-breed. To improve the overall strength of the method, it may be advantageous to use more than one biomarker. Thus, in an embodiment, method is performed for at least 5 of the miRNAs, such as for at least 10 of the miRNAs, preferably for at least 15 of the miRNAs, more preferably for at least 20 of the miRNAs and most preferably for all 24 biomarkers.

It may also be an advantage to include at least some of the listed biomarkers in the method based on the pathways they are involved in. As outlined in Table 3 in Example 4, the identified miRNAs have also been found to be involved in different pathways. In one embodiment, the method comprises determining the level of miRNAs that regulate the cell cycle. In an alternative embodiment, it may be advantageous to include miRNAs, which in combination are involved in two or more, such as three or more, preferably such as four or more or more preferably all of the pathways selected from the group consisting of cell cycle/cell cycle arrest, fatty acid synthesis, pain and stress, insulin signalling and reproduction. In a preferred embodiment, the miRNAs are involved in at least regulating the cell cycle—or regulate the cell cycle, more preferably cell cycle arrest.

The cell cycle is the series of events that takes place in a cell as it grows and divides into two genetically identical daughter cells. It is a highly regulated process that comprises several phases:
G1 Phase: Metabolic changes prepare the cell for division; the cell grows physically larger and organelles are copied.
S Phase: DNA is replicated.
G2 Phase: The cell grows more, makes proteins and organelles, and begins to reorganise its contents in preparation for mitosis.
M Phase: Nuclear DNA of the cell condenses into visible chromosomes and is pulled apart by the mitotic spindle, a specialised structure made out of microtubules. Mitosis takes place in five stages: prophase, prometaphase, metaphase, anaphase, and telophase.
Cytokinesis: The cytoplasm is split in two, resulting in two individual cells.

Cyclins and cyclin-dependent kinases (CDKs) are regulatory molecules that determine how a cell progresses through the cell cycle. When activated by a bound cyclin, CDKs phosphorylate target proteins to activate or deactivate them leading to coordinated progression through the cell cycle.

As used herein, “regulate” or “regulation” of the cell cycle refers to the mechanisms that control the orderly progression. Positive regulation of the cell cycle drives its progression, while negative regulation of the cell cycle slows progression or arrests the cell cycle.

Cell cycle arrest refers to a temporary or permanent halting of the cell cycle, preventing cells from progressing through the normal stages of cell division.

miRNAs can act as either positive or negative regulators of cell cycle progression. When miRNAs target and downregulate genes encoding cell cycle proteins, they can inhibit cell cycle progression and promote cell cycle arrest. Conversely, when miRNAs target genes encoding proteins that inhibit the cell cycle, they can promote cell cycle progression and proliferation. This delicate balance of miRNA mediated regulation is important for normal cell growth.

The cell cycle can be measured using a variety of commercially available assays. For example, DyeCycle stains can be used that measure DNA content distribution and are analysed with flow cytometry. Alternatively, antibodies for cell cycle analysis are also available, which can be analysed using flow cytometry or imaging.

In an embodiment, the one or more miRNAs are selected from the group listed in Table 3. The regulation is as indicated in Tables 1-2 and the corresponding text.

In another embodiment, the one or more miRNAs are selected from the group listed in Table 1, with the proviso that the cattle is Brown Swiss. The regulation is as indicated in Table 1 and the corresponding text.

In yet another embodiment, the one or more miRNAs are selected from the group listed in Table 2, with the proviso that the cattle is Holstein. The regulation is as indicated in Table 2 and the corresponding text.

Levels of miRNA can be determined using methods known to the person skilled in the art. Thus, in an embodiment, the levels are determined by a method selected from the group consisting of NGS, qPCR, dPCR, and ELISA miRNA.

### Determining Improvement

By comparing different samples obtained at different time points (minutes or hours), it may be possible to evaluate if any improvement in animal welfare has taken place. Thus, an aspect of the invention relates to a method for evaluating if at least one welfare parameter of cattle has improved, the method comprising:
determining in a first milk sample from said cattle, the level of one or more miRNAs selected from the group consisting of SEQ ID NO's: 94 2, 81, 134, 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 2, 81, 134, 85, 52, 74, 83, 6, 113, 70, 1, 69, 132, 120, 48, 112, 111, 31, 87 and 55;
determining in a second milk sample from said cattle, the level of one or more miRNAs selected from the group consisting of SEQ ID NO's: 94, 2, 81, 134, 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 2, 81, 134, 85, 52, 74, 83, 6, 113, 70, 1, 69, 132, 120, 48, 112, 111, 31, 87 and 55; wherein said second milk sample is obtained later in time than the first milk sample;
comparing said one or more levels in the first sample to the one or more corresponding levels in the second sample;
   wherein if said one or more corresponding levels of SEQ ID NO's: 94, 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 85, 52, 74, 83, 6, 113, 70, 1, 69, 132, 120, 48, 112, 111, 31, 87 and 55 in the second sample are equal to or above said levels in the first sample, it is indicative of that the welfare parameter has not improved; and
   wherein if said one or more corresponding levels of SEQ ID NO's: 94, 85, 52, 74, 83, 6, 113, 70, 1, 69, 132, 123, 120, 48, 112, 84, 111, 31, 87, 55 and 93 or a functional variant thereof wherein the functional variant has at least 60% sequence identity to SEQ ID NO's: 94, 123, 93, 84, 85, 52, 74, 83, 6, 113, 70, 1, 69, 132, 120, 48, 112, 111, 31, 87 and 55 in the second sample are lower than said levels in the first sample, it is indicative of that the welfare parameter has improved;
   wherein if said one or more corresponding levels of SEQ ID NO's: 2, 81 or 134 or a functional variant thereof wherein the functional variant has at least 60% overall sequence identity to SEQ ID NO: 2, 81 or 134 in the second sample are equal to or below said levels in the first sample, it is indicative of that the welfare parameter of the cattle has not improved; and
   wherein if said one or more corresponding levels of SEQ ID NO's: 2, 81 or 134 or a functional variant thereof wherein the functional variant has at least 60% overall sequence identity to SEQ ID NO: 2: 81, or 124 in the second sample are above said levels in the first sample, it is indicative of that the welfare parameter of the cattle has improved.

In an embodiment, initiatives have taken place between the sampling of the first sample and the sampling of the second sample, to improve the one or more welfare parameters of the cattle.

In yet an embodiment, the welfare parameter is heat stress.

Heat stress occurs when body temperature rises above normal due to an inability to dissipate heat effectively. This can happen when the environmental temperature is high, air movement is low, or humidity is high. Cattle rely on various mechanisms to regulate their body temperature, including panting, sweating, and behavioural changes. Signs and symptoms of heat stress in cattle include but are not limited to, an increased respiratory rate, increases salivation, loss of appetite, reduced milk production and increased susceptibility to disease.

TABLE 1
common parameters to determine heat stress:

| | Temperature | Respiration | Body |
|---|---|---|---|
| Heat Stress | Humdity Index | (Breaths per | Temperature |
| Severity | (THI) | minute) | (° C.) |
| No heat stress | Less than 68 | 40-60 | 38-39 |
| Mild | 68-71 | 60-75 | 39-39.5 |
| Mild to moderate | 72-79 | 75-85 | 39.5-40 |
| Moderate to severe | 80-90 | 85-100 | 40-40.5 |
| Severe | 90-99 | 100-104 | Over 40.5 |

In yet another embodiment, the initiatives are selected from the group consisting of a change in heat exposure, such as lowering of temperature, installation of fans, possibility of shadowing, installation of sprinklers, improved access to additional water, change in feed, reduction of the animal density, provision of cooling systems as appropriate for the local conditions, changes in moving cattle, and a less stressful environment.

In some embodiments there is a method of improving at least one of milk yield, milk quality, meat yield, meat quality from at least one head of cattle, the method comprising
i. determining in a first milk sample from the at least one head of cattle, the level of at least one miRNA as defined in SEQ ID NOs: 94, 2, 81, 134, 85, 52, 74, 83, 6, 113, 70, 1, 69, 132, 123, 120, 48, 112, 84, 111, 31, 87, 55, 93 or a functional variant thereof wherein the functional variant has at least 60% overall sequence identity to SEQ ID NO: 94, 2, 81, 134, 85, 52, 74, 83, 6, 113, 70, 1, 69, 132, 123, 120, 48, 112, 84, 111, 31, 87, 55 or 93;
ii. comparing the level of the at least one miRNA to a reference level; wherein where the level of the at least one miRNA is above said reference the welfare of the at least one head of cattle is compromised;
iii. improving the welfare of the at least one head of cattle;
iv. determining in a second milk sample from the same at least one head of cattle, the level of at least one miRNA as defined in SEQ ID NOs: 94, 2, 81, 134, 85, 52, 74, 83, 6, 113, 70, 1, 69, 132, 123, 120, 48, 112, 84, 111, 31, 87, 55, 93 or a functional variant thereof wherein the functional variant has at least 60% overall sequence identity to SEQ ID NO: 94, 2, 81, 134, 85, 52, 74, 83, 6, 113, 70, 1, 69, 132, 123, 120, 48, 112, 84, 111, 31, 87, 55 or 93; and
v. comparing the level in the first milk sample to the level in the second milk sample, wherein if the level of SEQ ID NOs: 94, 85, 52, 74, 83, 6, 113, 70, 1, 69, 132, 123, 120, 48, 112, 84, 111, 31, 87, 55 or 93 or a functional variant thereof wherein said functional variant has at least 60% overall sequence identity to the non-variant sequence in the second milk sample is lower than the level in the first milk sample or lower than the reference level at least one of milk yield, milk quality, meat yield and meat quality from at least one head of cattle the welfare of the head of cattle has improved and/or if the level of SEQ ID NOs: 2, 81 or 134 or a functional variant thereof with at least 60% overall sequence identity to the non-variant sequence in the second milk sample is higher than the level in the first milk sample or higher than the reference level at least one of milk yield, milk quality, meat yield and meat quality from at least one head of cattle the welfare of the head of cattle has improved.

Milk yield refers to the amount of milk produced by an animal over a given period of time. Improving milk yield means increasing the amount of milk produced in a given time period.

Meat yield refers to the amount of meat that can be obtained from an animal after slaughter. Meat yield is typically expressed as the percentage of the live weight or carcass weight of the animal.

Milk quality is a concept that encompasses multiple factors such as sensory qualities, composition and bacterial safety. High quality milk is a rich source of protein, fat, calcium and other nutrients such as branched chain fatty acids (BCA's). High quality milk is also free from contaminants, has a low bacterial count, is a rich creamy white colour with a clean fresh smell and a mild taste,

Meat quality is a complex concept that encompasses a variety of factors including:
Appearance: high quality meat has a bright appealing colour, uniform texture and no signs of discoloration.
Texture: high quality meat is tender and easy to chew, whereas low quality meat is chewy and/or tough.
Flavour: high quality meat has a richer and more complex flavour than low quality meat.

### Uses

Yet an aspect of the invention relates to the use of the level of one or more miRNAs selected from the group consisting of SEQ ID NO's: 94. 2, 81, 134, 85, 52, 74, 83, 6, 113, 70, 1, 69, 132, 123, 120, 48, 112, 84, 111, 31, 87, 55 and 93 in a cattle milk sample to evaluate the status of a welfare parameter of the cattle.

In an embodiment, the welfare parameter is heat stress.

### Device

The method of the invention may also be implemented in a device or system. Thus, yet another aspect of the invention relates to a device or system adapted to determine in a cattle milk sample or milk derived sample if at least one welfare parameter of cattle, preferably bovine, has been compromised or not, the device comprising:
a unit able to determine the level of one or more miRNAs in the milk sample or milk-derived sample;
a processor configured with a reference table, said reference table corresponding to reference levels of one or more of the miRNA according to Table 4;
   wherein the device or system is adapted to
receive the milk sample or milk-derived sample;
determine the levels of the one or more miRNAs;
comparing the determined levels of the one or more miRNAs to the reference table; and
determining if at least one welfare parameter of the cattle has been compromised or not.

In an embodiment, the reference table comprises corresponding miRNAs values from cattle where the corresponding welfare parameter has not been compromised.

In another embodiment, the device or system comprises a user interface, the user interface adapted to receive input from a user relating to one or more of, the miRNA tested, the breed from which the milk sample is derived, and the type of milk sample.

### Computer Implemented Method

The method of the invention can also be computer-implemented. Thus, a further aspect of the invention relates to a computer implemented method of determining in a cattle milk sample or milk-derived sample if at least one welfare parameter of cattle, preferably bovine, has been compromised or not, the method comprising:
providing levels of one or more miRNAs from a cattle milk sample or milk derived sample selected from Table 4;
providing a mathematical model comprising reference levels of one or more corresponding miRNAs selected from Table 4;
determining if the provided levels deviates (significantly) from the reference levels, using the mathematical model; and
providing to a system or a user a determination of at least one welfare parameter of the cattle being compromised or not.

In an embodiment, the reference table comprises corresponding miRNAs values from cattle where the corresponding welfare parameter has not been compromised.

Yet another aspect of the invention relates to a computer system comprising an input/output device and a processor, the system being enabled to execute the computer implemented method according to the invention on the processor.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—the Characterization of Physiological Parameters, Oxidative and Inflammatory Markers in Biologicals Samples from Cows of Two Breeds in Thermal Comfort Conditions and Heat Stress

### Aim of Study

To investigate changes of physiological parameters, oxidative and inflammatory markers in biologicals samples of Holstein (H) and Brown Swiss (BS) cows during temperature comfort conditions and heat stress.

### Materials and Methods

### Ethics Statement:

The trial carried out by the University of Milan during the summer of 2021 has been approved by the Ethical Committee for experiments with animals of the Department of Veterinary Medicine of the University of Bari.

### Experimental Conditions:

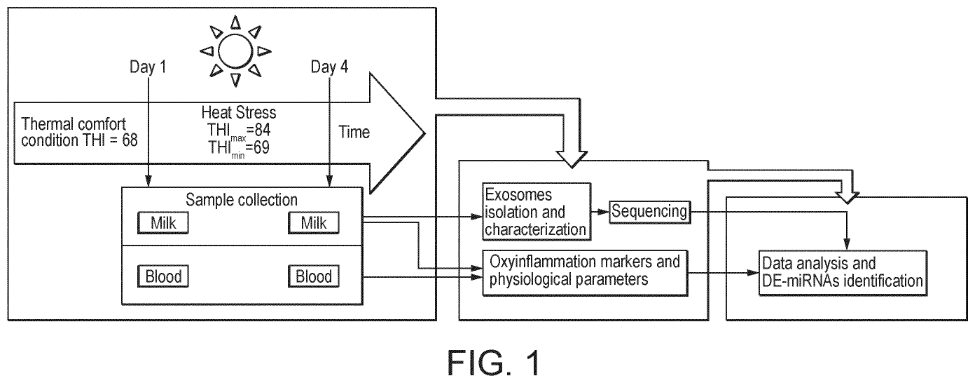

The Experimental design of the study is shown in FIG. 1. In vivo experiments investigating the adaptive responses of H and BS under farm conditions were carried out in the Apulia region during the summer of 2021. 18 multiparous mid-lactating H and 18 BS balanced for parity and days in milk (DIM—between 80 and 160 DIM) and reared in the same commercial farm were included in each group.

Environmental conditions were monitored by special equipment to measure the temperature-humidity index. The time-lapse video recording system was set to provide the collection of images during the whole trial to evaluate all the feeding, resting, and other primary activities and behavioural patterns. The lactating cows' barn was provided with fan coolers and automatic sprinklers in the feeding area and roof fans in the cubicle resting area.

During the warmest weeks of summer 2021, the cooling system was turned off for four consecutive days and then re-activated. Ambient temperature and relative humidity were recorded every 30 seconds across the trial period with Hobo Pro series Temp probes (Onset Computer Corp., Pocasset, MA, USA) to calculate THI to calculate the temperature-humidity index. The temperature-humidity index ranged from 68 to 86.

During the experimental period, cows were fed a standard total mixed ration. All physiological patterns were recorded daily, and biological samples were collected during the heat stress days (four days and then the other four after three days of cooling).

### Physiological Parameters:

All physiological data were collected at 4.00 am, 3.00 pm, and 9 pm on each of the four days of the trial (from day 1 to day 4).

Respiratory rate measurements were visually taken by expert people observing the movement of the animal's rib cage for 20 seconds. Then measures were multiplied by 3 to be quantified as breaths per minute (bpm).

Rectal temperatures were measured by a digital rectal thermometer. Eye, muzzle, skin, and vaginal temperature images were collected with a portable infrared thermography camera (ThermaCam i70 0, FLIR Systems AB, Danderyd, Sweden). To calibrate the camera results, environmental temperature and relative humidity were recorded with a digital thermo-hygrometer (Extech® 44550). To determine temperatures, an image analysis software Therma Cam Researcher Pro 2.8 SR-2 (FLIR Systems AB, Sweden), was used to measure the maximum temperature within an oval area traced around the anatomical site. This maximum temperature was included in the analyses. Two images were collected for each measurement, and the mean value of both photos was used for the analyses. Milk yield recording and milk sampling were performed during morning and evening milking sessions (at 4.00 am and 5.00 pm). From each cow, 100 ml of milk was sampled, and two percent 2-Bromo-2-nitro-1,2-propanediol was added as a preservative, refrigerated at 4° C., transferred to the laboratory, and analyzed within 2 h with near-infrared spectroscopy for fat, protein, lactose, dry matter, Urea, BHB (ISO 9622:2013 (IDF 141:2013). From these data, FCM yield, standardized at 4% fat, was calculated for each test-day record according to the following formula (Gaines, WL; Davidson, 1923):

$$4 \% FCM = 0.4 \times milk + 15 \times fat$$

Moreover, the energy-corrected milk (ECM) yield was calculated according to the formula reported (Yan et al., 2011):

$$E C M = milk \times [ 0.25 + ( 0.122 \times \% fat ) + ( 0.077 \times \% protein ) ]$$

### Blood Sampling and Analysis:

Blood samples were collected from each cow at 4.00 am, 3.00 pm, and 9 pm from day 1 to day 4. Blood was drawn from the coccygeal vein in sterile Vacutainer tubes (Becton Dickinson and Co.) 4 with lithium-heparin and clot activator tubes. All tubes were centrifuged on the farm, and serum and plasma were stored immediately at minus 20° C. until analysis.

Clinical biochemistry parameters were obtained from the serum samples using an automated biochemistry analyzer (CS-300B; Dirui, Changchun, China). The following parameters were assessed: alanine aminotransferase, aspartate aminotransferase, creatine phosphokinase, lactate dehydrogenase, alkaline phosphatase, glucose, blood urea nitrogen, creatinine, total serum protein, albumin, cholesterol, triglycerides, non-esterified fatty acids, calcium, phosphate, magnesium, chloride (Gesan Production Kit, Campobello di Mazara, Trapani, Italy). Globulins and Albumin/Globulin ratio were calculated starting from total protein and albumin parameters. The multi-parameter analyzer was calibrated using specific standards (Seracal, Gesan Production Kit, Campobello di Mazara, Trapani, Italy). After setting the calibration curve, two multi-parameter control sera (Seracontrol N and Seracontrol P, Gesan Production Kit, Campobello di Mazara, Trapani, Italy) were used to verify internal accuracy, which was lower than 3.00% in compliance with the manufacturer's declared values. Each sample was analyzed in triplicate, and their mean was used for further analysis.

### Plasma Oxidative Profile and Total Antioxidant Capacity:

Plasma (0.5 ml) was placed in a 50-mL test tube and homogenized with 15 mL of deionized distilled water (DDW). Homogenate (1 mL) was transferred to a glass tube for the Thiobarbituric acid reactive substances (TBARS) determination, and 0.05 mL of butylated hydroxytoluene (7.2% in ethanol) was added along with 1.950 ml of thiobarbituric acid (TBA)/trichloroacetic acid (TCA)/HCl (0.375% TBA, 15% TCA, and 0.25 N HCl). The sample solution was shaken and then incubated at 90° C. for 15 min. in a thermostatic bath. Samples were cooled to room temperature (15-30° C.) and then centrifuged at 2000×g for 15 min. Supernatant absorbance (λ_{531 nm}) was measured; the negative control solution was TBA/TCA/HCl (2 ml+1 ml DDW). The TBARS were calculated using a standard curve designed with 1,1,3,3-tetra methoxy propane, and the concentration of lipid oxidation was expressed as milligrams of malondialdehyde per 1 ml of plasma.

For hydroperoxides, 0.5 ml of plasma were added with 4 mL of CH₃OH and 2 mL of CHCl3. Samples were vortexed for 30 s, and 2 mL of CHCl3 and 1.6 mL of 0.9% NaCl were added, vortexed for 1 min. and then centrifuged at 3500×g for 10 min. at 4° C. Two millilitres of lipid extract were sampled from the lower chloroform phase and then processed with 1 mL of CH₃COOH/CHCl₃ and 50 μL of KI (1.2 g/L ml distilled water). Samples were stored for 5 min. in a dark room, 3 mL of 0.5% of CH₃COOCd were added, vortexed, and centrifuged at 4500×g for 10 min. at 40° C. Absorbance (λ_{353 nm}) was measured against a blank tube in which plasma was replaced by 2 mL of distilled water. The results were expressed in micromoles per ml.

Plasma (0.5 ml) was placed in 20 mL of 0.15 M KCl for 2 min. Two aliquots of homogenate (50 μL each) were added with 1 mL 10% TCA and then centrifuged at 1200×g for 3 min. at 4° C. to measure protein oxidation. The first aliquot was used as a standard, and 1 mL of 2 M HCl solution was added. The second aliquot was added with 1 mL of 2 M HCl containing 10 mM 2,4-dinitrophenyl hydrazine (DNPH). The samples were incubated for 1 h at room temperature (15 to 30° C.) and vortexed every 20 min. Then, 1 mL of 10% TCA was added. The samples were vortexed for 30 s and centrifuged three times at 1200×g for 3 min. at 4° C., and the supernatant was removed. The pellet was washed with 1 mL of ethanol:ethyl acetate (1:1), shaken, and centrifuged three times at 1200×g for 3 min. at 4° C., and the supernatant was removed. The pellet was then dissolved in 1 mL 20 mM sodium phosphate 6 M guanidine hydrochloride buffer. The samples were then shaken and centrifuged at 1200×g for 3 min. at 4° C. Carbonyl concentration was calculated on the DNPH treated sample at 360 nm with a Beckman Coulter DU800 (Beckman Instruments Inc., Brea, CA, USA) and expressed as nanomoles carbonyl per milligrams protein. Protein concentration was calculated according to the Biuret assay.

The total antioxidant capacity (FRAP) assay was used to measure total antioxidant potential as described (Benzie and Strain, 1996), with a slight modification. Three mL of freshly prepared FRAP reagent (1 mL of a 10 mM TPTZ solution in 40 mM HCl plus 1 mL of 20 mM FeCl3 in 10 mL H2O solution and 10 ml of 300 mM acetate buffer, pH 3.6) was incubated at 37° C. for 40 min after mixing with 100 μL of plasma sample or supernatant. The absorbance of the reaction mixture was recorded (λ734_{593 nm}), and the antioxidant power was expressed as μmol Trolox equivalents/ml. 2,2′-Azino-bis [3-ethylbenzothiazoline-6-sulphonic acid] (ABTS) radical scavenging activity was determined according to the procedure previously described (Re et al., 1999) with some modifications. Briefly, ABTS radical cation was produced by mixing 7 mM ABTS stock solution with 2.45 mM potassium persulfate and keeping the mixture in the dark at 25° C. for 12 to 16 h. The solution was then diluted in PBS to reach an absorbance value of 0.70±0.02 at 734 nm. Then, 10 μL of plasma sample was added to 990 μL of diluted ABTS radical cation solution and incubated at 30° C. for 5 min. The reagent blank was prepared by adding 10 μL of PBS instead of the sample. The scavenging of the ABTS radical cation was determined spectrophotometrically (λ_{734 nm}).

Antioxidant activity was expressed as percentage inhibition of ABTS radical cation and calculated by the following equation:

$$\% inhibition = 100 \times ( Absorbance734 Control - Absorbance734 Sample ) \text{} / \text{} Absorbance734 \text{} Control .$$

Each sample was analyzed in triplicate.

### Statistical Analysis:

Statistical analysis was conducted in SAS v. 9.4 (SAS Institute, Cary, NC, USA, 2011). Shapiro-Wilk tests of the collected data revealed no deviation from normality for all the stress variables used (results not shown), so parametric statistics were used. Continuous data (i.e., physiological and milk parameters) were analyzed by generalized linear mixed models using PROC MIXED with fixed effects of breed, time (day or daily time as repeated measures), and their interaction. Blood parameters were analyzed by generalized linear mixed models using PROC MIXED with fixed effects of breed, time (considering only first blood sampling as thermal comfort condition and last blood sample as heat stress), and their interaction. Significance was set at P<0.05.

### Results

1. Physiological parameters. Both breeds (H and BS) showed the same trend during the experimental trial. Cows showed higher values of rectal temperature, respiration rate, and vaginal and skin temperature, with values that rising in line with the higher temperature-humidity index values during the day and falling during the night, when the temperature-humidity index values were at their lowest (data not shown).
2. Milk yield, fat-corrected milk, and energy-corrected milk are affected negatively by heat stress (data not shown).
3. Fat percentage, the concentrations of protein, and casein are affected by heat stress (data not shown).
4. The serum protein profile showed differences due to heat stress. Total proteins were higher in both breeds; Moreover, blood urea nitrogen and uric acid were affected by heat stress and breed. In detail, BUN increased after heat stress in both breeds, while uric acid decreased (data not shown).
5. The electrolyte serum profile was partially affected by heat stress; the chloride concentration increased in H after heat stress, the phosphorous concentration increased, and the amount of magnesium decreased after heat stress (data not shown).
6. The serum lipid profile is affected heat stress. The cholesterol was lower in H, but heat stress did not affect its amount, while triglycerides decreased after heat stress in both breeds, showing similar values. Not esterified fatty acids were higher in BS compared with H in thermal comfort conditions but decreased after heat stress reaching similar values (data not shown).
7. Heat stress and breeds did not affect the serum enzymatic profile (data not shown).
8. Heat shock protein 70 increased in both breeds after heat stress. Similarly, serum amyloid proteins decreased with heat stress; haptoglobin decreased in H and increased in BS after heat stress (data not shown).
9. The oxidative profile was affected by breed and heat stress. The thiobarbituric acid reactive substances values decreased after heat stress in BS and increased in H, showing the opposite trend, while hydroperoxides and protein carbonyls increased only in BS (data not shown).

### Conclusion

The physiological parameters investigated confirmed that cows of both breeds suffered from heat stress and dissipate heat similarly. Quantitative and qualitative milk parameters were influenced by heat stress.

### Example 2—the Analysis of miRNA Profile of Milk-Derived Exosomes Using Next Generation Sequencing in Milk from Cows of Two Breeds in Thermal Comfort Conditions and Heat Stress

### Aim of the Study

To investigate microRNA profile in milk of single animal milk-derived exosomes collected from Holstein (H) and Brown Swiss (BS) cows during temperature comfort conditions and heat stress.

### Materials and Methods

Experimental conditions and collection of the milk samples:

See Example 1.

### Extracellular Vesicles (Exosomes) Isolation:

Extracellular Vesicles (EVs) were purified from 10 ml of milk by ultracentrifugation and Size Exclusion Chromatography (SEC). Skimmed milk was centrifuged at 10,000 g for 30 min at 4° C. to remove remaining fat and cellular debris. The supernatant was transferred to the ultracentrifuge tubes and ultracentrifuged at 100,000 g for 1 h at 4° C. using a fixed rotor (Beckman Coulter TY65 fixed angle rotor, Pasadena, USA). The exosome pellet was carefully collected on top of the casein pellet and deposited in 2 mL tubes without disturbing it. The collected exosomes were re-suspended and further purified through SEC, using the qEVoriginal 35 nm columns (Izon), following the manufacturer's instructions. After the void volume (3 mL), 4 fractions of 500 μL each were collected. Fractions 2 and 3, expected to contain the exosomes, were pooled. The column equilibration and the fractions' elution were carried out using sterile three times-filtered (0.22 μm) Ammonium Bicarbonate buffer 20 mM pH 7.5.

### Exosomes Characterization:

### 1. Nanoparticle Tracking Analysis

The size and concentration of EVs were assessed immediately after using the NanoSight NS300 (Malvern Panalytical). The purified exosomes were diluted 50 or 100 times using fresh three-times filtered PBS (0.22 μm). Particles were visualized and analyzed by the NTA 3.3 Dev Build 3.3.301 software. The instrument set up was to operate at 22° C., syringe pump speed 30 AU, and for each sample were recorded 5 videos of 60 sec each; results were the mean of the 5 measurements.

### 2. Transmission Electron Microscopy (TEM)

To assess the morphology, exosomes were visualized using negative staining by TEM. A few microliters of samples were absorbed on glow-discharged carbon-coated formvar copper grids contrasted with 2% uranyl acetate, air-dried, and observed in an FEI Talos 120 kV transmission electron microscope (FEI Company, Netherlands). Images of exosomes were acquired by a 4 k×4K Ceta CMOS camera.

### 3. Western Blot Analysis

The milk exosome protein concentration was determined by the Pierce Bicinchoninic acid (BCA) protein assay kit (ThermoScientific, Illinois, USA). Exosomal proteins (2 μg) were loaded on sodium dodecyl sulfate-polyacrylamide electrophoresis (SDS-PAGE) gel and Western blotted on nitrocellulose membrane, using Trans-Blot Turbo Midi 0.2 μm Nitrocellulose Transfer Packs (Bio-Rad Laboratories, California, USA) and the Trans-Blot Turbo Transfer System (Bio-Rad Laboratories). The membrane was blocked for 1 h with ROTIRBlock 1× (Carlroth, Cat. No. A151.1), incubated with primary antibodies anti-CD9 (Biorad, MCA469GT, 1:1500) overnight at 4° C. and then with the secondary antibody polyclonal anti-mouse peroxidase (Dako, P0260, 1:2000) for 1 h at room temperature; or with the primary antibody anti-TSG101 (Abcam, ab225877, 1:2000) overnight at 4° C. and secondary antibody polyclonal anti-rabbit peroxidase (Vector, PI-1000, 1:3000) for 1 h at room temperature. Immunoreactive bands were visualized by enhanced chemiluminescence (Millipore, Cat. No. WBKLS0050).

EVs miRNA Extraction and Sequencing:

Small RNAs were extracted using a microRNA concentrator kit (A&A Biotechnology, Cat. No 035-25) following the manufacturers' instructions. Only EVs eluted in the second and third fractions after the SEC. The RNA quality and quantity were verified according to MIQE guidelines (Bustin et al., 2009). For all samples, RNA concentration was quantified by Qubit® 2.0 Fluorometer with Qubit® microRNA Assay Kit (Invitrogen, Cat. No. Q32880). Small RNA transcripts were converted into barcoded cDNA libraries. Library preparation was performed as previously reported (Pardini et al., 2018) using the NEBNext Multiplex small RNA Library Prep Set (Cat. No. cat number NEB #E7560) for Illumina and run on the NextSeq500 (Illumina Inc., USA). Samples from H and BS were loaded and mixed on the flow cells to mix to balance thermal comfort conditions and heat stress.

The output of the NextSeq500 Illumina sequencer was demultiplexed using bcl2fastq Illumina software embedded in the docker4seq package (Cordero et al., 2012) (Pardini et al., 2018). MiRNA expression quantification was performed using the previously described workflow (Pardini et al., 2018) using the implementation described (Beccuti et al., 2018). Sequences were mapped using SHRIMP (Rumble et al., 2009) to Bos taurus precursors miRNAs available in miRBase 22.1 (http://www.mirbase.org/). Counts tables were used to perform differential expression (DE) analysis using as threshold an adjusted P-value≤0.1 and an absolute log 2 Fold Change (log 2FC)≥1. CPM (counts per million reads; tables were used for heat maps generation and PCA (data not shown).

miRNA Target Prioritization:

The target genes of DE-miRNAs were predicted using MiRWalk 3.0 (Sticht et al., 2018), which includes 3 miRNA-target prediction programs (miRDB (Wong and Wang, 2015), miRTarBase (Hsu et al., 2011) and Targetscan (Agarwal et al., 2015). The analysis targeted the entire gene sequence (5′UTR, CDS, and 3′UTR). The list of target genes predicted by the three tools was included in further analysis. Functional mRNA enrichment was performed using DAVID (Database for Annotation, Visualization, and Integrated Discovery) bioinformatic resource (Huang et al., 2009a, 2009b) and biological pathways in the KEGG (Kyoto Encyclopedia of Genes and Genomes) (Kanehisa et al., 2012) were examined for enrichment.

### Statistical Analysis:

Statistical analysis was carried out using XLStat for Windows (Addinsoft, New York, U.S.A.), IBM SPSS Statistics 25 software (IBM Corp., 2017), and MedCalc 14.0 (MedCalc Software bvba, Ostend, Belgium). Differential expression (DE) analysis was performed using DESeq2 (Ferrero et al., 2018), implemented in docker4seq. DE analysis was performed using paired samples (i.e., day 1 and day 4 of the same animal). Statistical significance was accepted at p<0.05. P-values were adjusted using the Bonferroni correction.

Hierarchical clustering (Euclidean distance, average linkage, only gene clustering, after Z-score transformation of log 2CPM) was performed to identify heat stress-specific signature. A Principal Component Analysis (PCA) was performed using the cluster analysis R package using the log 2CPM data.

### Results

### Extracellular Vesicle Characterization

The EVs from the milk of thermal comfort condition—and heat stress—cows (6 animals, 3 H, and 3 BS) were isolated by ultracentrifugation and size exclusion chromatography. The particle size distribution was assessed by nanoparticle tracking analysis (NTA), revealing that the EV population was characterized by small vesicles. The modal average of the six cows was 136.15 nm±23.74 nm and 139.6 nm±22.7 nm, and the concentration was 4.56E+11 and 1.5E+11 in thermal comfort condition and heat stress, respectively.

The modal average of thermal comfort conditions and heat stress EVs was 131.53 nm±26.95 nm and 134.95 nm±33.17 nm for H, and 140.8 nm±24.87 and 144.2 nm±11.1 nm for BS at thermal comfort conditions and heat stress, respectively. The concentration of isolated EVs was 6.92E+11 particles/ml and 1.03 E+11 particles/ml for H at thermal comfort conditions and heat stress, respectively, and 2.15 E+11 particles/ml and 1.98 E+11 particles/ml for BS at thermal comfort conditions and heat stress, respectively. No statistical differences were identified in size or concentration.

The shape and integrity of EVs were assessed by electronic transmission microscopy, showing the presence of whole, undamaged small EVs. Western blot analysis was performed to examine the expression of different exosomal markers in milk-derived exosomes of thermal comfort conditions and heat stress cows. Two different exosomal markers, one expressed on the membrane of the exosomes, the tetraspanin CD9, and the other delivered in the lumen of EVs, the TSG101, were identified.

Identification of miRNAs Mapped Using Bos taurus Precursor

After RNA extraction, small RNAs were sequenced on the NextSeq500 sequencer (Illumina). Many reads per sample were obtained, varying from 2,053,467 (sample h16d1) to 130,228,352 (sample h2978d4). Mapping on miRNA precursors (miRbase 22) and miRNA quantification was performed using the miRNA pipeline implemented in docker4seq, and Quality Control was performed with MultiQC. The relatively low mapping is expected because small RNAs were extracted from milk EVs.

### Conclusion

The analysis revealed the expression of 1998 and 1882 Bos taurus miRNAs in the milk EVs of H and BS, respectively, discarding lowly expressed miRNAs (baseMean≤1).

### Example 3—the Identification of miRNA Expressed Differently in Milk from Cows of Two Breeds in Heat Stress

### Aim of the Study

To investigate microRNA expressed differently in milk from Holstein (H) and Brown Swiss (BS) cows during temperature comfort conditions and heat stress.

### Materials and Methods

Experimental conditions and collection of the milk samples as described in Example 1. For differential expression analysis (DE) results of the 1998 and 1882 Bos taurus miRNAs revealed in the Example 2 were used.

Differential expression analysis between miRNA in milk from cows in thermal comfort conditions and in heat stress:

Differential expression (DE) analysis was performed using DESeq2, implemented in docker4seq. DE analysis was performed using paired samples (i.e., day 1 and day 4 of the same animal), and results are provided in the NGS summary. Hierarchical clustering (Euclidean distance, average linkage, only gene clustering, after Z-score transformation of log₂CPM) was performed to identify heat stress-specific signature.

### Results

### Brown Swiss:

To determine the differences in the exo-miRNAs expression profile of thermal comfort conditions—and heat stressed milk, a differential expression (DE) analysis using DESeq2, with a threshold adjusted P-value<0.1 and |log 2FC|>1, was performed. A difference in miRNA profiles was observed, suggesting molecular changes due to heat stress.

Comparing the milk-exosome profiles in thermal comfort conditions and heat stress, 132 miRNAs were significantly altered. The level of 80 miRNAs increased (1 to 2.44 log₂FC) after heat stress and of 52 decreased (−1 to −1.64 log₂FC) after heat stress (Table 2).

TABLE 2
132 milk-derived miRNAs was identified to be significantly deregulated in Brown
swiss under heat stress.

| SEQ ID | | log₂Fold | |
|---|---|---|---|
| NO | miRNA name | change | miRNA sequence 5′→3′ |
| 1 | bta-let-7b-3p | 1.0808412 | uuccgucauccaacauaucaa |
| 2 | bta-miR-10165-3p | −1.5167605 | ccggagacgucuacgacuccacggug |
| 3 | bta-miR-10166-5p | −1.0799857 | gugcagcugaucacagacugug |
| 4 | bta-miR-10167-3p | 1.61512935 | cggguggucggggcgggucag |
| 5 | bta-miR-10174-3p | 1.02381625 | aucacauugccagggauuaccacg |
| 6 | bta-miR-10225b-5p | 1.41938258 | ugugaccugccaggcacugccuug |
| 7 | bta-miR-103-1-3p | 1.23068238 | agcagcauuguacagggcuauga |
| 8 | bta-miR-103-2-3p | 1.19718426 | agcagcauuguacagggcuauga |
| 9 | bta-miR-107-3p | 1.05967079 | agcagcauuguacagggcuauc |
| 10 | bta-miR-11973-3p | 1.09517943 | cuugcugagugaccucccugcu |
| 11 | bta-miR-11976-1-3p | 1.21265882 | ggcggcggcggcgccggggcg |
| 12 | bta-miR-11976-2-3p | 1.07845865 | ggcggcggcggcgccggggcg |
| 13 | bta-miR-11989-5p | −1.1700179 | cccagggauguagcuccuagugc |
| 14 | bta-miR-11991-3p | −1.208694 | guacaaucaagauauagaacau |
| 15 | bta-miR-12002a-5p | −1.3787873 | caaccaaagauuucacaug |
| 16 | bta-miR-12031-3p | −1.0128688 | auuggagccagucugcagaagc |
| 17 | bta-miR-12034-3p | 1.89853694 | ccccggggagcccggcggu |
| 18 | bta-miR-12036-3p | 1.03471494 | gucccgggcgccgcggagccu |
| 19 | bta-miR-12054-5p | 1.32765943 | ggggucuuuacaaag |
| 20 | bta-miR-12060-3p | −1.2299903 | agccuucucccaugugucuccucc |
| 21 | bta-miR-1249-3p | 1.06748999 | acgcccuucccccccuucuuca |
| 22 | bta-miR-1260b-5p | 1.54476481 | aucccaccacugccacca |
| 23 | bta-miR-127-5p* | −1.1676555 | agccugcugaagcucagagggcucuga |
| 24 | bta-miR-1306-5p | 1.4056291 | ccaccuccccugcaaacgucc |
| 25 | bta-miR-1307-3p | 1.21053478 | acucggcguggcgucggucgug |
| 26 | bta-miR-140-3p | 1.08225772 | uaccacaggguagaaccacgga |
| 27 | bta-miR-142-3p | −1.2876828 | aguguuuccuacuuuauggaug |
| 28 | bta-miR-142-5p | −1.002588 | cauaaaguagaaagcacuac |
| 29 | bta-miR-1603-5p* | −1.228734 | aaaccucacucauaaccagcauccca |
| 30 | bta-miR-16a-5p | 1.0524393 | uagcagcacguaaauauuggug |
| 31 | bta-miR-1777a-3p* | 2.08242361 | ccggccucugggcccag |
| 32 | bta-miR-181b-2-5p | 1.03103514 | aacauucauugcugucgguggguu |
| 33 | bta-miR-184-3p | −1.0473481 | uggacggagaacugauaagggu |
| 34 | bta-miR-185-5p | 1.21018204 | uggagagaaaggcaguuccuga |
| 35 | bta-miR-197-3p | 1.30521915 | uucaccaccuucuccacccagc |
| 36 | bta-miR-20a-5p | 1.2656834 | uaaagugcuuauagugcagguag |
| 37 | bta-miR-210-3p | 1.12597687 | acugugcgugugacagcggcuga |
| 38 | bta-miR-219-2-3p | −1.0298283 | agaguugagucuggacgucccg |
| 39 | bta-miR-2284aa-2-3p* | −1.5400061 | aaaaccugaaacaaacuuuuua |
| 40 | bta-miR-2284i-3p* | 1.5234776 | aaacccggaauuaacuuuuu |
| 41 | bta-miR-2284v-5p* | −1.5085941 | guucuccaaggaagacugugu |
| 42 | bta-miR-2284w-3p* | −1.181307 | gaaaaaccucaaugaacucuuu |
| 43 | bta-miR-2284z-2-5p | −1.1315055 | aaaaaaguuuguuuggguuuuu |
| 44 | bta-miR-2285as-3-3p | −1.3443056 | aagcccugaaugaacuuuuugg |
| 45 | bta-miR-2285bu-1-5 | −1.2732525 | aaaaaggucguucggguuuu |
| 46 | bta-miR-2285ck-5p | −1.6260266 | uggccaaaaucuuuguuucagu |
| 47 | bta-miR-2285m-4-5p | −1.0792486 | ccaaaaauuucguucagguuuu |
| 48 | bta-miR-2285n-1-5p | 2.71352755 | ccaaaaaguucgucccgguguuu |
| 49 | bta-miR-2285n-4-5p | −1.1856787 | cuaaaaaguucauuugguuuuuu |
| 50 | bta-miR-22850-3-5p | −1.0806786 | caaaaaauuuguuuggguuu |
| 51 | bta-miR-2285z-5p | −1.0499513 | ccagaaaguucauucagguccu |
| 52 | bta-miR-2288-3p | 2.17336678 | agguaguaggugugugguuu |
| 53 | bta-miR-2292-3p | −1.0098674 | gagucugagauugaggaaggca |
| 54 | bta-miR-2311-5p | −1.2293256 | uacugaaacugugcucguggugu |
| 55 | bta-miR-2326-3p | 1.05676041 | ccccccuuccucuggaaaaa |
| 56 | bta-miR-2340-3p | 1.32339107 | ggacuucccugguggucuugug |
| 57 | bta-miR-2344-5p | 1.10571866 | gauuccugucccucgaggugguuc |
| 58 | bta-miR-2360-5p | 1.08058192 | agcgccaccagugcgccugcuccg |
| 59 | bta-miR-2364-3p | −1.1197783 | cugcucgcccgaccccagagu |
| 60 | bta-miR-2367-5p | −1.3082584 | acccuguaacucagccaucagag |
| 61 | bta-miR-2382-3p | 1.41349285 | cuacuuccuggugccucgccccac |
| 62 | bta-miR-2390-3p | −1.0980648 | ggagagaaagagcaauuucagg |
| 63 | bta-miR-2394-3p | −1.0179237 | agccugagaggaaaggggaau |
| 64 | bta-miR-23b-3p | 1.15573653 | aucacauugccagggauuaccac |
| 65 | bta-miR-2404-2-5p | −1.0366101 | uugcacugcaugguaucugc |
| 66 | bta-miR-2409-3p | −1.6450517 | ucucacauggacgcugcccu |
| 67 | bta-miR-2410-3p | 1.36978029 | ucccugugcuucccugugguccag |
| 68 | bta-miR-2416-5p | −1.2957812 | caguccgaagacacccuaccuac |
| 69 | bta-miR-2427-3p | 1.28046492 | gggcuuaucacugugacca |
| 70 | bta-miR-2436-3p | 1.83369102 | cccgcccgaccgccucgcccaa |
| 71 | bta-miR-2446-3p | −1.0537007 | gaccaauuucaaguauauuuuc |
| 72 | bta-miR-2467-3p | −1.0248462 | gagggcagugaggcucagggcug |
| 73 | bta-miR-2488-3p | −1.1704339 | uagaauuaagggaa |
| 74 | bta-miR-25-5p | 1.05043168 | agaggcggagacuugggcaauug |
| 75 | bta-miR-2885-3p | 1.10965946 | cggcggcagcgccggggcg |
| 76 | bta-miR-2888-1-3p | 1.2974013 | ggugggguggggggguugg |
| 77 | bta-miR-2892-3p | 1.12420519 | ggggacaguccgccccgccccgcc |
| 78 | bta-miR-2895-3p | −1.0360668 | gaaauaucugagugucuguuuu |
| 79 | bta-miR-2897-5p | 1.37607626 | ggugggagggucccaccgag |
| 80 | bta-miR-29d-5p | 1.32941558 | ugaccgauuucuccugguguu |
| 81 | bta-miR-301b-5p | −1.1842433 | gugcucugacgagguugcacuacugug |
| 82 | bta-miR-302b-5p | −1.040572 | uaacuuuaacaugguaguacuuu |
| 83 | bta-miR-30a-5p | 1.19616021 | uguaaacauccucgacuggaagcu |
| 84 | bta-miR-30b-5p | 2.44378236 | uguaaacauccuacacucagcu |
| 85 | bta-miR-30c-5p | 1.44935174 | uguaaacauccuacacucucagc |
| 86 | bta-miR-30d-5p | 1.1143995 | uguaaacauccccgacuggaagcu |
| 87 | bta-miR-30f-5p | 1.72981798 | uguaaacacccuacacucucagcu |
| 88 | bta-miR-324-3p | 1.69651314 | acccacugccccaggugcugcu |
| 89 | bta-miR-326-3p | 1.33797131 | ccucugggcccuuccuccag |
| 90 | bta-miR-328-3p | 1.05314833 | cuggcccucucugcccuuccgu |
| 91 | bta-miR-331-3p | 1.25595294 | gccccugggccuauccuagaa |
| 92 | bta-miR-339a-3p | 1.02654918 | gugcgcuccucgaggccagagc |
| 93 | bta-miR-339a-5p | 1.71345795 | ucccuguccuccaggagcucac |
| 94 | bta-miR-339b-5p | 1.53557183 | ucccuguccuccaggagcuc |
| 95 | bta-miR-342-3p | 1.15143134 | ucucacacagaaaucgcacccaucu |
| 96 | bta-miR-3604-1-3p | −1.1595182 | gguagucugaacacuggguu |
| 97 | bta-miR-361-3p | 1.1183531 | gucccccaggugugauucugauuu |
| 98 | bta-miR-365-1-3p | 1.33885711 | uaaugccccuaaaaauccuuau |
| 99 | bta-miR-365-2-3p | 1.40679543 | uaaugccccuaaaaauccuuau |
| 100 | bta-miR-375-5p | 1.02766189 | cgcgacgagccccucgcacaaac |
| 101 | bta-miR-409a-5p | 1.86354305 | agguuacccgagcaacuuugcau |
| 102 | bta-miR-425-5p | 1.15713384 | augacacgaucacucccguuga |
| 103 | bta-miR-4286-1-5p | 1.45628063 | accccacuccugguacc |
| 104 | bta-miR-4286-2-3p | 1.77312498 | accccacuccugguacc |
| 105 | bta-miR-449a-5p | −1.1355674 | uggcaguguauuguuagcuggu |
| 106 | bta-miR-4680-5p | −1.0822569 | agaacucuugcagucuuagaugu |
| 107 | bta-miR-484-5p | 1.29393604 | ucaggcucaguccccucccgau |
| 108 | bta-miR-487a-5p | −1.1042527 | agugguuaucccugcuguguu |
| 109 | bta-miR-494-5p | −1.2465659 | gagguuauccguguugucuuc |
| 110 | bta-miR-500-3p | 1.13110769 | uaauccuugcuaccugggugaga |
| 111 | bta-miR-500-5p | 1.70622187 | ugcaccugggcaaggauuc |
| 112 | bta-miR-502b-5p | 2.00202395 | aauucuugcccaggugagagu |
| 113 | bta-miR-532-3p | 1.75413033 | acccucccacacccaaggcuug |
| 114 | bta-miR-544a-5p | −1.4352993 | gaucuuguuaaaaggcagauu |
| 115 | bta-miR-6123-3p | 1.01571701 | ccuuugagcugggaagggaa |
| 116 | bta-miR-631-3p | −1.0715707 | gcugauagacugagucaggg |
| 117 | bta-miR-6517-3p | −1.0045776 | gccggaacuccggguccug |
| 118 | bta-miR-652-3p | 1.44005637 | aauggcgccacuaggguugug |
| 119 | bta-miR-6523a-5p | 1.24391448 | ucugggguaacuuugagcaggg |
| 120 | bta-miR-6525-5p | 1.89676673 | cuggggaaagcaggagugag |
| 121 | bta-miR-6529b-5p | −1.0465888 | agagguaaaaaggcacag |
| 122 | bta-miR-6530-3p | −1.2044249 | cugugcuugcggcucccgcg |
| 123 | bta-miR-664b-3p | 1.67828397 | uauucauuuaucucccagccuac |
| 124 | bta-miR-669-5p | 1.45393176 | ugugggugugugcaugugcgug |
| 125 | bta-miR-670-3p | −1.0481232 | gguuuccucauauucauucaggag |
| 126 | bta-miR-670-5p | −1.0134385 | ucccugaguauauguggugaa |
| 127 | bta-miR-6715-5p | 1.18215655 | acaggcacggccaguuugagc |
| 128 | bta-miR-677-3p | −1.2719695 | guuugcugaagccagaugccauuucugag |
| 129 | bta-miR-877-5p | 1.26038463 | guagaggagauggcgcaggg |
| 130 | bta-miR-885-5p | 1.15300381 | uccauuacacuacccugccucu |
| 131 | bta-miR-92a-1-3p | 1.19076671 | uauugcacuugucccggccugu |
| 132 | bta-miR-92a-2-3p | 1.17674585 | uauugcacuugucccggccugu |

### Holstein:

To determine the differences in the exo-miRNAs expression profile of thermal comfort conditions—and heat stressed milk, a differential expression (DE) analysis using DESeq2, with a threshold adjusted P-value<0.1 and |log₂FC|>1, was performed. A difference in miRNA profiles was observed, suggesting molecular changes due to heat stress.

Comparing the milk-exosome profiles in thermal comfort conditions and heat stress, 32 miRNAs were significantly altered. The level of 25 miRNAs increased (1 to 2.24 log₂FC) after heat stress and 7 decreased (−1 to −1.33 log₂FC) after heat stress (Table 3).

TABLE 3
25 milk-derived miRNAs was identified to be significantly deregulated in Holstein
under heat stress.

| SEQ ID | | log₂Fold | |
|---|---|---|---|
| NO | Name | Change | miRNA sequence |
| 1 | bta-let-7b-3p | 1.02545438 | uuccgucauccaacauaucaa |
| 2 | bta-miR-10165-3p | 1.2619762 | ccggagacgucuacgacuccacggug |
| 133 | bta-miR-10166-3p | 1.05744816 | gugcagcugaucacagacugug |
| 6 | bta-miR-10225b-5p | 1.40237231 | ugugaccugccaggcacugccuug |
| 134 | bta-miR-11980-5p | −1.0812932 | aggcaacgggcuuggcggag |
| 135 | bta-miR-12053-5p | 1.39118552 | uguagaacauguuuuggaac |
| 31 | bta-miR-1777a-3p | 1.22644972 | ccggccucugggcccag |
| 136 | bta-miR-181b-1-3p | −1.029657 | agcucacugaacaaugagugca |
| 137 | bta-miR-2285di-3p | 1.22257846 | aaacccugaaggaacauuuuggu |
| 48 | bta-miR-2285n-1-5p | 1.76270571 | ccaaaaaguucgucccgguguuu |
| 138 | bta-miR-2285u-5p | 1.09178751 | aauguucauuccaauuuuuc |
| 52 | bta-miR-2288-3p | 2.24547928 | agguaguaggugugugguuu |
| 139 | bta-miR-2293-5p | −1.0305487 | ugauuuuguuguuuuguauu |
| 55 | bta-miR-2326-3p | 1.0840075 | ccccccuuccucuggaaaaa |
| 140 | bta-miR-2400-5p | −1.0027906 | cugcccgagcgcauguggcuga |
| 69 | bta-miR-2427-3p | 1.01980171 | gggcuuaucacugugacca |
| 70 | bta-miR-2436-3p | 1.27283655 | cccgcccgaccgccucgcccaa |
| 74 | bta-miR-25-5p | 1.55401273 | agaggcggagacuugggcaauug |
| 141 | bta-miR-2889-3p | −1.2869097 | accggggagggggccggggauc |
| 81 | bta-miR-301b-5p | −1.3340818 | gugcucugacgagguugcacuacugug |
| 83 | bta-miR-30a-5p | 1.08162423 | uguaaacauccucgacuggaagcu |
| 84 | bta-miR-30b-5p | 2.03132024 | uguaaacauccuacacucagcu |
| 85 | bta-miR-30c-5p | 1.18532277 | uguaaacauccuacacucucagc |
| 87 | bta-miR-30f-5p | 1.41210764 | uguaaacacccuacacucucagcu |
| 93 | bta-miR-339a-5p | 1.34015241 | ucccuguccuccaggagcucac |
| 94 | bta-miR-339b-5p | 1.21013191 | ucccuguccuccaggagcuc |
| 111 | bta-miR-500-5p | 1.36064556 | ugcaccugggcaaggauuc |
| 112 | bta-miR-502b-5p | 1.38474999 | aauccaccugggcaaggauuc |
| 113 | bta-miR-532-3p | 1.43747655 | acccucccacacccaaggcuug |
| 120 | bta-miR-6525-5p | 2.02240186 | cuggggaaagcaggagugag |
| 123 | bta-miR-664b-3p | 1.44223631 | uauucauuuaucucccagccuac |
| 132 | bta-miR-92a-2-3p | 1.0302931 | uauugcacuugucccggccugu |

### Conclusion

132 miRNAs for Brown Swiss and 32 miRNAs for Holstein cows were revealed as altered in milk by heat stress.

### Example 4—the Identification of miRNA Expressed Similarly in Milk from Cow of Two Breeds in Heat Stress

### Aim of the Study

To investigate microRNA expressed similarly in milk from Holstein (H) and Brown Swiss (BS) cows during heat stress conditions.

### Materials and Methods

Experimental conditions and collection of the milk samples described in Example 1. To identify the common heat stress signature between two breeds, the Venn analysis was performed for results from the Example 3.

### Results

Results demonstrated that 24 miRNAs are dysregulated in both BS and H. The level of 3 differently expressed miRNAs decreased after heat stress, while the level of 21 increased after heat stress (Table 4).

TABLE 4
24 miRNAs in milk identified to be deregulated in both Holstein and Brown swiss
under heat stress.

| | | | |
|---|---|---|---|
| SEQ ID | | | |
| NO | Name | miRNA sequence | Pathway |

Decreased (n = 3)

| | | | |
|---|---|---|---|
| 2 | bta-miR-10165- | ccggagacgucuacgacuccacggug | No data reported |
| | 3p | | |
| 81 | bta-miR-301b- | gugcucugacgagguugcacuacugug | Cell cycle/cell cycle |
| | 5p | | arrest |
| | | | Fatty acid synthesis |
| 134 | bta-miR-11980- | aggcaacgggcuuggcggag | Cell cycle/cell cycle |
| | 5p | | arrest |

Increased (n = 21)

| | | | |
|---|---|---|---|
| 85 | bta-miR-30c-5p | uguaaacauccuacacucucagc | Cell cycle/cell cycle |
| | | | arrest |
| | | | Fatty acid synthesis |
| | | | Pain and stress |
| 52 | bta-miR-2288- | agguaguaggugugugguuu | No data reported |
| | 3p | | |
| 74 | bta-miR-25-5p | agaggcggagacuugggcaauug | Cell cycle/cell cycle |
| | | | arrest |
| | | | Insulin signalling |
| | | | pathway |
| | | | Reproduction |
| | | | Pain and stress |
| 83 | bta-miR-30a-5p | uguaaacauccucgacuggaagcu | Cell cycle/cell cycle |
| | | | arrest |
| | | | Reproduction |
| | | | Pain and stress |
| 6 | bta-miR- | ugugaccugccaggcacugccuug | No data reported |
| | 10225b-5p | | |
| 113 | bta-miR-532-3p | acccucccacacccaaggcuug | Cell cycle/cell cycle |
| | | | arrest |
| 70 | bta-miR-2436- | cccgcccgaccgccucgcccaa | No data reported |
| | 3p | | |
| 1 | bta-let-7b-3p | uuccgucauccaacauaucaa | Cell cycle/cell cycle |
| | | | arrest |
| | | | Insulin signalling |
| | | | pathway |
| | | | Pain and stress |
| | | | Fatty acid synthesis |
| 69 | bta-miR-2427- | gggcuuaucacugugacca | No data reported |
| | 3p | | |
| 132 | bta-miR-92a-2- | uauugcacuugucccggccugu | Cell cycle/cell cycle |
| | 3p | | arrest |
| | | | Insulin signalling |
| | | | pathway |
| | | | Reproduction |
| | | | Fatty acid synthesis |
| 123 | bta-miR-664b- | uauucauuuaucucccagccuac | Cell cycle/cell cycle |
| | 3p | | arrest |
| | | | Reproduction |
| 120 | bta-miR-6525- | cuggggaaagcaggagugag | No data reported |
| | 5p | | |
| 48 | bta-miR-2285n- | ccaaaaaguucgucccgguguuu | No data reported |
| | 1-5p | | |
| 112 | bta-miR-502b- | aauucuugcccaggugagagu | Cell cycle/cell cycle |
| | 5p | | arrest |
| 84 | bta-miR-30b-5p | uguaaacauccuacacucagcu | Cell cycle/cell cycle |
| | | | arrest |
| | | | Fatty acid synthesis |
| 111 | bta-miR-500-5p | ugcaccugggcaaggauuc | Cell cycle/cell cycle |
| | | | arrest |
| | | | Pain and stress |
| 31 | bta-miR-1777a- | ccggccucugggcccag | No data reported |
| | 3p | | |
| 87 | bta-miR-30f-5p | uguaaacacccuacacucucagcu | No data reported |
| 55 | bta-miR-2326- | ccccccuuccucuggaaaaa | No data reported |
| | 3p | | |
| 93 | bta-miR-339a- | ucccuguccuccaggagcucac | Cell cycle/cell cycle |
| | 5p | | arrest |
| 94 | bta-miR-339b- | ucccuguccuccaggagcuc | Cell cycle/cell cycle |
| | 5p | | arrest |
| | | | Pain and stress |

### Conclusion

The data shows that surprisingly 24 miRNAs have been identified which are deregulated across different bovine breeds under heat stress. Thus, biomarkers have been identified which does not need to take the breed into account, when determining/evaluating whether dairy cattle has been exposed to heat stress.

The invention will now be further described with reference to the following clauses:

Clause 1. A method for evaluating at least one welfare parameter of cattle, preferably bovine, the method comprising:
determining in a milk sample from said cattle, the level of one or more miRNAs selected from the group consisting of SEQ ID NO's: 2, 81, 85, 52, 74, 83, 6, 113, 70, 1, 69, 132, 123, 120, 48, 112, 84, 111, 31, 87, 55, 93, and 94;
comparing said one or more levels to one or more corresponding reference levels;
   wherein if said one or more miRNA levels of SEQ ID NO's: 85, 52, 74, 83, 6, 113, 70, 1, 69, 132, 123, 120, 48, 112, 84, 111, 31, 87, 55, 93, and 94 are above said one or more reference levels, it is indicative of that at least one welfare parameter of the cattle has been compromised; and
   wherein if said one or more miRNA levels of SEQ ID NO's: 85, 52, 74, 83, 6, 113, 70, 1, 69, 132, 123, 120, 48, 112, 84, 111, 31, 87, 55, 93, and 94 are equal to or below said one or more reference levels, it is indicative of that the at least one welfare parameter of the cattle has not been compromised;
   wherein if said one or more miRNA levels of SEQ ID NO's: 2, 81 are equal to or above said one or more reference levels, it is indicative of that the at least one welfare parameter of the cattle has not been compromised; and
   wherein if said one or more miRNA levels of SEQ ID NO's: 2, 81 are below said one or more reference levels, it is indicative of that the at least one welfare parameter of the cattle has been compromised.

Clause 2. The method according to clause 1, wherein the at least one compromised welfare parameter is caused by a parameter selected from the group consisting of heat stress, social stress, metabolic stress, disease stress, and combinations thereof, preferably the parameter is heat stress.

Clause 3. The method according to clause 1 or 2, wherein the compromised welfare parameter is caused by heat stress.

Clause 4. The method according to any of the preceding clauses, wherein the cattle is dairy cattle.

Clause 5. The method according to any of the preceding clauses, wherein said milk sample is from a single cattle or a mixed milk sample from a herd of cattle.

Clause 6. The method according to any of the preceding clauses 1-4, wherein said milk sample is from a herd of cattle.

Clause 7. The method according to any of the preceding clauses, wherein the level of miRNA, is the level of miRNA in milk-derived exosomes.

Clause 8. The method according to any of the preceding clauses, wherein the method is performed for at least 5 of the miRNAs, such as for at least 10 of the miRNAs, preferably for at least 15 of the miRNAs, more preferably for at least 20 of the miRNAs and most preferably for all 23 biomarkers.

Clause 9. The method according to any of the preceding clauses, wherein said reference level is the level of the one or more miRNA's in a milk sample from a cattle having normal welfare or an average level from several dairy having normal welfare.

Clause 10. A method for evaluating if at least one welfare parameter of cattle has improved, the method comprising:
determining in a first milk sample from said cattle, the level of one or more miRNAs selected from the group consisting of SEQ ID NO's: 2, 81, 85, 52, 74, 83, 6, 113, 70, 1, 69, 132, 123, 120, 48, 112, 84, 111, 31, 87, 55, 93, and 94;
determining in a second milk sample from said cattle, the level of one or more miRNAs selected from the group consisting of SEQ ID NO's: 2, 81, 85, 52, 74, 83, 6, 113, 70, 1, 69, 132, 123, 120, 48, 112, 84, 111, 31, 87, 55, 93, and 94; wherein said second milk sample is obtained later in time than the first milk sample;
comparing said one or more levels in the first sample to the one or more corresponding levels in the second sample;
   wherein if said one or more corresponding levels of SEQ ID NO's: 85, 52, 74, 83, 6, 113, 70, 1, 69, 132, 123, 120, 48, 112, 84, 111, 31, 87, 55, 93, and 94 in the second sample are equal to or above said levels in the first sample, it is indicative of that the welfare parameter has not improved; and
   wherein if said one or more corresponding levels of SEQ ID NO's: 85, 52, 74, 83, 6, 113, 70, 1, 69, 132, 123, 120, 48, 112, 84, 111, 31, 87, 55, 93, and 94 in the second sample are lower than said levels in the first sample, it is indicative of that the welfare parameter has improved;
   wherein if said one or more corresponding levels of SEQ ID NO's: 2 and 81 in the second sample are equal to or below said levels in the first sample, it is indicative of that the welfare parameter of the cattle has not improved; and
   wherein if said one or more corresponding levels of SEQ ID NO's: 2 and 81 in the second sample are above said levels in the first sample, it is indicative of that the welfare parameter of the cattle has improved.

Clause 11. The method according to clause 10, wherein initiatives has been taken between the sampling of the first sample and the sampling of the second sample, to improve the one or more welfare parameters of the cattle, such as wherein the initiatives are selected from the group consisting of change in heat exposure, such as lowering of temperature, installation of fans, possibility of shadowing, installation of sprinklers, improved access to additional water, change in feed, reduction of the animal density, provision of cooling systems as appropriate for the local conditions, changes in moving cattle, and less stressful environment.

Clause 12. Use of the level of one or more miRNAs selected from the group consisting of SEQ ID NO's: 2, 81, 85, 52, 74, 83, 6, 113, 70, 1, 69, 132, 123, 120, 48, 112, 84, 111, 31, 87, 55, 93, and 94 in a cattle milk sample to evaluate the status of a welfare parameter of the cattle.

Clause 13. The use according to clause 12, wherein the welfare parameter is heat stress.

Clause 14. A device or system adapted to determine in a cattle milk sample or milk derived sample if at least one welfare parameter of cattle, preferably bovine, has been compromised or not, the device comprising:
a unit able to determine the level of one or more miRNAs in the milk sample or milk derived sample;
a processor configured with a reference table, said reference table corresponding to reference levels of one or more of the miRNA according to Table 4;
   wherein the device or system is adapted to
receive the milk sample milk derived sample;
determine the levels of the one or more miRNAs;
comparing the determined levels of the one or more miRNAs to the reference table; and
determining if at least one welfare parameter of the cattle has been compromised or not.

Clause 15. A computer implemented method of determining in a cattle milk sample or milk derived sample if at least one welfare parameter of cattle, preferably bovine, has been compromised or not, the method comprising:
providing levels of one or more miRNAs from a cattle milk sample or milk derived sample selected from Table 4;
providing a mathematical model comprising reference levels of one or more corresponding miRNAs selected from Table 4;
determining if the provided levels deviates significantly from the reference levels, using the mathematical model; and
providing to a system or a user a determination of at least one welfare parameter of the cattle has been compromised or not.

1. A method of determining at least one welfare parameter of at least one head of cattle, the method comprising:
i. determining, in a milk sample, a level of at least one miRNA that regulates, at least, the cell cycle, wherein the at least one miRNA is selected from SEQ ID NO: 94, 123, 93, 84, 81, 134, 85, 74, 83, 113, 1, 132, 112, and 111 or a functional variant thereof, wherein the functional variant has at least 60% overall sequence identity to SEQ ID NO: 94, 123, 93, 84, 81, 134, 85, 74, 83, 113, 1, 132, 112, and 111 respectively; and
ii. comparing the level of the at least one miRNA to a reference level, wherein if the level of the at least one miRNA selected from SEQ ID NO: 94, 123, 93, 84, 85, 74, 83, 113, 1, 132, 112, and 111 is above the reference level at least one welfare parameter of the at least one head of cattle has been or is compromised or if the level of the at least one miRNA selected from SEQ ID NO: 81 and 134 is below the reference level at least one welfare parameter of the at least one head of cattle has been or is compromised.

2. A method of improving at least one welfare parameter of at least one head of cattle, the method comprising:
i. determining, in a first milk sample, a level of at least one miRNA that regulates, at least, the cell cycle, wherein the at least one miRNA is selected from SEQ ID NO: 94, 123, 93, 84, 81, 134, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111 respectively;
ii. comparing the level of the at least one miRNA to a reference level, wherein if the level of the at least one miRNA selected from SEQ ID NO: 94, 123, 93, 84, 85, 74, 83, 113, 1, 132, 112, and 111 is above the reference level at least one welfare parameter of the at least one head of cattle has been compromised or if said the level of the at least one miRNA selected from SEQ ID NO: 81 and 134 is below the reference level at least one welfare parameter of the at least one head of cattle has been compromised;
iii. improving welfare of the at least one head of cattle;
iv. determining, in a second milk sample, the level of the at least one miRNA selected from SEQ ID NO: 94, 123, 93, 84, 81, 134, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111 respectively; and
v. comparing the level of the at least one miRNA in the first milk sample to the level of the at least one miRNA in the second milk sample, wherein if the level of SEQ ID NO: 94, 123, 93, 84, 85, 74, 83, 113, 1, 132, 112, and 111 or 55 or a functional variant thereof wherein the functional variant has at least 60% sequence identity to SEQ ID NO: 94, 123, 93, 84, 85, 74, 83, 113, 1, 132, 112, and 111 respectively in the second milk sample is lower than the level in the first milk sample or lower than the reference level the welfare of the at least one head of cattle has improved; or if the level of SEQ ID NO: 81 or 134 in the second milk sample is higher than the level in the first milk sample or higher than the reference level the welfare of the at least one head of cattle has improved.

3. A method of improving at least one of milk yield, milk quality, meat yield or meat quality of at least one head of cattle, the method comprising:
i. determining, in a first milk sample, a level of at least one miRNA that regulates, at least, the cell cycle, wherein the at least one miRNA is selected from SEQ ID NO: 94, 123, 93, 84, 85, 74, 83, 113, 1, 132, 112, and 111 or a functional variant thereof wherein the functional variant has at least 60% sequence identity to SEQ ID NO: 94, 123, 93, 84, 81, 134, 85, 74, 83, 113, 1, 132, 112, and 111;
ii. comparing the level of the at least one miRNA to a reference level, wherein if the level of the at least one miRNA selected from SEQ ID NO: 94, 123, 93, 84, 85, 74, 83, 113, 1, 132, 112, and 111 is above the reference level at least one welfare parameter of the at least one head of cattle has been or is compromised or if the level of the at least one miRNA selected from SEQ ID NO: 81 and 134 is below the reference level at least one welfare parameter of the at least one head of cattle has been compromised; and
iii. improving welfare of the at least one head of cattle; wherein improving the welfare of the at least one head of cattle improves at least one of milk yield, milk quality, meat yield and meat quality.

4. A method of determining at least one welfare parameter of at least one head of cattle, the method comprising:
determining a level of at least one miRNA, wherein the at least one miRNA is selected from SEQ ID NO: 94, 123, 93, 84, 2, 81, 134, 85, 52, 74, 83, 6, 113, 70, 1, 69, 132, 120, 48, 112, 111, 31, 87 and 55 or a functional variant thereof, wherein said functional variant has at least 60% overall sequence identity to SEQ ID NO: 94, 123, 93, 84, 2, 81, 134, 85, 52, 74, 83, 6, 113, 70, 1, 69, 132, 120, 48, 112, 111, 31, 87 and 55 respectively;
wherein when the level of SEQ ID NO: 94, 123, 93, 84, 85, 52, 74, 83, 6, 113, 70, 1, 69, 132, 120, 48, 112, 111, 31, 87 and 55 or a functional variant thereof is increased by 1 log 2FC or more compared to a reference level or wherein the level of SEQ ID NO: 2, 81 or 134 is decreased by −1 log 2FC or more compared to a reference level, at least one welfare parameter of the at least one head of cattle has been or is not compromised.

5. The method according to claim 3, wherein the cattle is dairy cattle.

6. The method of claim 3, wherein the breed of cattle is Holstein or Brown Swiss.

7. The method according to claim 3, wherein the milk sample is from a herd of cattle.

8. The method according to claim 3, wherein the welfare parameter of the at least one head of cattle may be or is comprised by at least one of heat stress, social stress, metabolic stress, disease stress or combinations thereof.

9. The method according to claim 8, wherein the welfare of the at least one head of cattle has been or is compromised by heat stress.

10. The method according to claim 1, wherein the method comprises determining the level of all of SEQ ID NO: 94, 123, 93, 84, 85, 74, 83, 113, 1, 132, 112, and 111 or a functional variant thereof wherein the functional variant has at least 60% sequence identity to SEQ ID NO's: 94, 123, 93, 84, 81, 134, 85, 74, 83, 113, 1, 132, 112, and 111 respectively.

11. The method according to claim 3, wherein (i) the reference level is the level of the one or more miRNA in a reference milk sample from at least one reference head of cattle where the welfare of the at least one reference head of cattle is not or has not been compromised or (ii) the reference level is an average level from cattle where the welfare of the cattle is not or has not been compromised.

12. The method according to claim 2 wherein improving the welfare of the at least one head of cattle comprises one or more of changing heat exposure, improving access to additional water, changing the feed, reducing animal density and the provision of cooling systems.

13. A device adapted to determine if at least one welfare parameter of at least one head of cattle has been or is compromised, the device comprising:
i. a unit able to determine the level of one or more miRNAs according to claim 1;
ii. a processor configured with a reference table, the reference table corresponding to reference levels of one or more of the miRNA according to Table 4;
iii, wherein the device is adapted to
iv. receive the milk sample;
v. determine the levels of the one or more miRNAs;
vi. compare the determined levels of the one or more miRNAs to the reference table; and
vii. determine if the welfare of the at least one head of cattle has been or is compromised.

14. A computer implemented method of determining if welfare of at least one head of cattle has been compromised, the method comprising:
i. providing levels of one or more miRNAs from a cattle milk sample selected from Table 4;
ii. providing a mathematical model comprising reference levels of one or more corresponding miRNAs selected from Table 4;
iii. determining if the provided levels deviate significantly from the reference levels, using the mathematical model; and
iv. providing to a system or a user a determination of whether the welfare of the at least one head of cattle has been or is compromised.

15. A kit for evaluating at least one welfare parameter of at least one head of cattle, the kit comprising the device of claim 15 and further comprising instructions for use.

16. The method according to claim 3, wherein the cattle is bovine dairy cattle.


SEQUENCE LISTING (141 sequences)

SEQ ID NO: 1 - RNA, 21 nt, Bos taurus
  source 1..21: mol_type = other RNA
ttccgtcatccaacatatcaa

SEQ ID NO: 2 - RNA, 26 nt, Bos taurus
  source 1..26: mol_type = other RNA
ccggagacgtctacgactccacggtg

SEQ ID NO: 3 - RNA, 22 nt, Bos taurus
  source 1..22: mol_type = other RNA
gtgcagctgatcacagactgtg

SEQ ID NO: 4 - RNA, 21 nt, Bos taurus
  source 1..21: mol_type = other RNA
cgggtggtcggggcgggtcag

SEQ ID NO: 5 - RNA, 24 nt, Bos taurus
  source 1..24: mol_type = other RNA
atcacattgccagggattaccacg

SEQ ID NO: 6 - RNA, 24 nt, Bos taurus
  source 1..24: mol_type = other RNA
tgtgacctgccaggcactgccttg

SEQ ID NO: 7 - RNA, 23 nt, Bos taurus
  source 1..23: mol_type = other RNA
agcagcattgtacagggctatga

SEQ ID NO: 8 - RNA, 23 nt, Bos taurus
  source 1..23: mol_type = other RNA
agcagcattgtacagggctatga

SEQ ID NO: 9 - RNA, 22 nt, Bos taurus
  source 1..22: mol_type = other RNA
agcagcattgtacagggctatc

SEQ ID NO: 10 - RNA, 22 nt, Bos taurus
  source 1..22: mol_type = other RNA
cttgctgagtgacctccctgct

SEQ ID NO: 11 - RNA, 21 nt, Bos taurus
  source 1..21: mol_type = other RNA
ggcggcggcggcgccggggcg

SEQ ID NO: 12 - RNA, 21 nt, Bos taurus
  source 1..21: mol_type = other RNA
ggcggcggcggcgccggggcg

SEQ ID NO: 13 - RNA, 23 nt, Bos taurus
  source 1..23: mol_type = other RNA
cccagggatgtagctcctagtgc

SEQ ID NO: 14 - RNA, 22 nt, Bos taurus
  source 1..22: mol_type = other RNA
gtacaatcaagatatagaacat

SEQ ID NO: 15 - RNA, 19 nt, Bos taurus
  source 1..19: mol_type = other RNA
caaccaaagatttcacatg

SEQ ID NO: 16 - RNA, 22 nt, Bos taurus
  source 1..22: mol_type = other RNA
attggagccagtctgcagaagc

SEQ ID NO: 17 - RNA, 19 nt, Bos taurus
  source 1..19: mol_type = other RNA
ccccggggagcccggcggt

SEQ ID NO: 18 - RNA, 21 nt, Bos taurus
  source 1..21: mol_type = other RNA
gtcccgggcgccgcggagcct

SEQ ID NO: 19 - RNA, 15 nt, Bos taurus
  source 1..15: mol_type = other RNA
ggggtctttacaaag

SEQ ID NO: 20 - RNA, 24 nt, Bos taurus
  source 1..24: mol_type = other RNA
agccttctcccatgtgtctcctcc

SEQ ID NO: 21 - RNA, 22 nt, Bos taurus
  source 1..22: mol_type = other RNA
acgcccttcccccccttcttca

SEQ ID NO: 22 - RNA, 18 nt, Bos taurus
  source 1..18: mol_type = other RNA
atcccaccactgccacca

SEQ ID NO: 23 - RNA, 27 nt, Bos taurus
  source 1..27: mol_type = other RNA
agcctgctgaagctcagagggctctga

SEQ ID NO: 24 - RNA, 21 nt, Bos taurus
  source 1..21: mol_type = other RNA
ccacctcccctgcaaacgtcc

SEQ ID NO: 25 - RNA, 22 nt, Bos taurus
  source 1..22: mol_type = other RNA
actcggcgtggcgtcggtcgtg

SEQ ID NO: 26 - RNA, 22 nt, Bos taurus
  source 1..22: mol_type = other RNA
taccacagggtagaaccacgga

SEQ ID NO: 27 - RNA, 22 nt, Bos taurus
  source 1..22: mol_type = other RNA
agtgtttcctactttatggatg

SEQ ID NO: 28 - RNA, 20 nt, Bos taurus
  source 1..20: mol_type = other RNA
cataaagtagaaagcactac

SEQ ID NO: 29 - RNA, 26 nt, Bos taurus
  source 1..26: mol_type = other RNA
aaacctcactcataaccagcatccca

SEQ ID NO: 30 - RNA, 22 nt, Bos taurus
  source 1..22: mol_type = other RNA
tagcagcacgtaaatattggtg

SEQ ID NO: 31 - RNA, 17 nt, Bos taurus
  source 1..17: mol_type = other RNA
ccggcctctgggcccag

SEQ ID NO: 32 - RNA, 24 nt, Bos taurus
  source 1..24: mol_type = other RNA
aacattcattgctgtcggtgggtt

SEQ ID NO: 33 - RNA, 22 nt, Bos taurus
  source 1..22: mol_type = other RNA
tggacggagaactgataagggt

SEQ ID NO: 34 - RNA, 22 nt, Bos taurus
  source 1..22: mol_type = other RNA
tggagagaaaggcagttcctga

SEQ ID NO: 35 - RNA, 22 nt, Bos taurus
  source 1..22: mol_type = other RNA
ttcaccaccttctccacccagc

SEQ ID NO: 36 - RNA, 23 nt, Bos taurus
  source 1..23: mol_type = other RNA
taaagtgcttatagtgcaggtag

SEQ ID NO: 37 - RNA, 23 nt, Bos taurus
  source 1..23: mol_type = other RNA
actgtgcgtgtgacagcggctga

SEQ ID NO: 38 - RNA, 22 nt, Bos taurus
  source 1..22: mol_type = other RNA
agagttgagtctggacgtcccg

SEQ ID NO: 39 - RNA, 22 nt, Bos taurus
  source 1..22: mol_type = other RNA
aaaacctgaaacaaacttttta

SEQ ID NO: 40 - RNA, 20 nt, Bos taurus
  source 1..20: mol_type = other RNA
aaacccggaattaacttttt

SEQ ID NO: 41 - RNA, 21 nt, Bos taurus
  source 1..21: mol_type = other RNA
gttctccaaggaagactgtgt

SEQ ID NO: 42 - RNA, 22 nt, Bos taurus
  source 1..22: mol_type = other RNA
gaaaaacctcaatgaactcttt

SEQ ID NO: 43 - RNA, 22 nt, Bos taurus
  source 1..22: mol_type = other RNA
aaaaaagtttgtttgggttttt

SEQ ID NO: 44 - RNA, 22 nt, Bos taurus
  source 1..22: mol_type = other RNA
aagccctgaatgaactttttgg

SEQ ID NO: 45 - RNA, 20 nt, Bos taurus
  source 1..20: mol_type = other RNA
aaaaaggtcgttcgggtttt

SEQ ID NO: 46 - RNA, 22 nt, Bos taurus
  source 1..22: mol_type = other RNA
tggccaaaatctttgtttcagt

SEQ ID NO: 47 - RNA, 22 nt, Bos taurus
  source 1..22: mol_type = other RNA
ccaaaaatttcgttcaggtttt

SEQ ID NO: 48 - RNA, 23 nt, Bos taurus
  source 1..23: mol_type = other RNA
ccaaaaagttcgtcccggtgttt

SEQ ID NO: 49 - RNA, 23 nt, Bos taurus
  source 1..23: mol_type = other RNA
ctaaaaagttcatttggtttttt

SEQ ID NO: 50 - RNA, 20 nt, Bos taurus
  source 1..20: mol_type = other RNA
caaaaaatttgtttgggttt

SEQ ID NO: 51 - RNA, 22 nt, Bos taurus
  source 1..22: mol_type = other RNA
ccagaaagttcattcaggtcct

SEQ ID NO: 52 - RNA, 20 nt, Bos taurus
  source 1..20: mol_type = other RNA
aggtagtaggtgtgtggttt

SEQ ID NO: 53 - RNA, 22 nt, Bos taurus
  source 1..22: mol_type = other RNA
gagtctgagattgaggaaggca

SEQ ID NO: 54 - RNA, 23 nt, Bos taurus
  source 1..23: mol_type = other RNA
tactgaaactgtgctcgtggtgt

SEQ ID NO: 55 - RNA, 20 nt, Bos taurus
  source 1..20: mol_type = other RNA
ccccccttcctctggaaaaa

SEQ ID NO: 56 - RNA, 22 nt, Bos taurus
  source 1..22: mol_type = other RNA
ggacttccctggtggtcttgtg

SEQ ID NO: 57 - RNA, 24 nt, Bos taurus
  source 1..24: mol_type = other RNA
gattcctgtccctcgaggtggttc

SEQ ID NO: 58 - RNA, 24 nt, Bos taurus
  source 1..24: mol_type = other RNA
agcgccaccagtgcgcctgctccg

SEQ ID NO: 59 - RNA, 21 nt, Bos taurus
  source 1..21: mol_type = other RNA
ctgctcgcccgaccccagagt

SEQ ID NO: 60 - RNA, 23 nt, Bos taurus
  source 1..23: mol_type = other RNA
accctgtaactcagccatcagag

SEQ ID NO: 61 - RNA, 24 nt, Bos taurus
  source 1..24: mol_type = other RNA
ctacttcctggtgcctcgccccac

SEQ ID NO: 62 - RNA, 22 nt, Bos taurus
  source 1..22: mol_type = other RNA
ggagagaaagagcaatttcagg

SEQ ID NO: 63 - RNA, 21 nt, Bos taurus
  source 1..21: mol_type = other RNA
agcctgagaggaaaggggaat

SEQ ID NO: 64 - RNA, 23 nt, Bos taurus
  source 1..23: mol_type = other RNA
atcacattgccagggattaccac

SEQ ID NO: 65 - RNA, 20 nt, Bos taurus
  source 1..20: mol_type = other RNA
ttgcactgcatggtatctgc

SEQ ID NO: 66 - RNA, 20 nt, Bos taurus
  source 1..20: mol_type = other RNA
tctcacatggacgctgccct

SEQ ID NO: 67 - RNA, 24 nt, Bos taurus
  source 1..24: mol_type = other RNA
tccctgtgcttccctgtggtccag

SEQ ID NO: 68 - RNA, 23 nt, Bos taurus
  source 1..23: mol_type = other RNA
cagtccgaagacaccctacctac

SEQ ID NO: 69 - RNA, 19 nt, Bos taurus
  source 1..19: mol_type = other RNA
gggcttatcactgtgacca

SEQ ID NO: 70 - RNA, 22 nt, Bos taurus
  source 1..22: mol_type = other RNA
cccgcccgaccgcctcgcccaa

SEQ ID NO: 71 - RNA, 22 nt, Bos taurus
  source 1..22: mol_type = other RNA
gaccaatttcaagtatattttc

SEQ ID NO: 72 - RNA, 23 nt, Bos taurus
  source 1..23: mol_type = other RNA
gagggcagtgaggctcagggctg

SEQ ID NO: 73 - RNA, 14 nt, Bos taurus
  source 1..14: mol_type = other RNA
tagaattaagggaa

SEQ ID NO: 74 - RNA, 23 nt, Bos taurus
  source 1..23: mol_type = other RNA
agaggcggagacttgggcaattg

SEQ ID NO: 75 - RNA, 19 nt, Bos taurus
  source 1..19: mol_type = other RNA
cggcggcagcgccggggcg

SEQ ID NO: 76 - RNA, 19 nt, Bos taurus
  source 1..19: mol_type = other RNA
ggtggggtgggggggttgg

SEQ ID NO: 77 - RNA, 24 nt, Bos taurus
  source 1..24: mol_type = other RNA
ggggacagtccgccccgccccgcc

SEQ ID NO: 78 - RNA, 22 nt, Bos taurus
  source 1..22: mol_type = other RNA
gaaatatctgagtgtctgtttt

SEQ ID NO: 79 - RNA, 20 nt, Bos taurus
  source 1..20: mol_type = other RNA
ggtgggagggtcccaccgag

SEQ ID NO: 80 - RNA, 20 nt, Bos taurus
  source 1..20: mol_type = other RNA
tgaccgatttctcctggtgt

SEQ ID NO: 81 - RNA, 27 nt, Bos taurus
  source 1..27: mol_type = other RNA
gtgctctgacgaggttgcactactgtg

SEQ ID NO: 82 - RNA, 23 nt, Bos taurus
  source 1..23: mol_type = other RNA
taactttaacatggtagtacttt

SEQ ID NO: 83 - RNA, 24 nt, Bos taurus
  source 1..24: mol_type = other RNA
tgtaaacatcctcgactggaagct

SEQ ID NO: 84 - RNA, 22 nt, Bos taurus
  source 1..22: mol_type = other RNA
tgtaaacatcctacactcagct

SEQ ID NO: 85 - RNA, 23 nt, Bos taurus
  source 1..23: mol_type = other RNA
tgtaaacatcctacactctcagc

SEQ ID NO: 86 - RNA, 24 nt, Bos taurus
  source 1..24: mol_type = other RNA
tgtaaacatccccgactggaagct

SEQ ID NO: 87 - RNA, 24 nt, Bos taurus
  source 1..24: mol_type = other RNA
tgtaaacaccctacactctcagct

SEQ ID NO: 88 - RNA, 22 nt, Bos taurus
  source 1..22: mol_type = other RNA
acccactgccccaggtgctgct

SEQ ID NO: 89 - RNA, 20 nt, Bos taurus
  source 1..20: mol_type = other RNA
cctctgggcccttcctccag

SEQ ID NO: 90 - RNA, 22 nt, Bos taurus
  source 1..22: mol_type = other RNA
ctggccctctctgcccttccgt

SEQ ID NO: 91 - RNA, 21 nt, Bos taurus
  source 1..21: mol_type = other RNA
gcccctgggcctatcctagaa

SEQ ID NO: 92 - RNA, 22 nt, Bosavirus bosa
  source 1..22: mol_type = other RNA
gtgcgctcctcgaggccagagc

SEQ ID NO: 93 - RNA, 22 nt, Bos taurus
  source 1..22: mol_type = other RNA
tccctgtcctccaggagctcac

SEQ ID NO: 94 - RNA, 20 nt, Bos taurus
  source 1..20: mol_type = other RNA
tccctgtcctccaggagctc

SEQ ID NO: 95 - RNA, 25 nt, Bos taurus
  source 1..25: mol_type = other RNA
tctcacacagaaatcgcacccatct

SEQ ID NO: 96 - RNA, 20 nt, Bos taurus
  source 1..20: mol_type = other RNA
ggtagtctgaacactgggtt

SEQ ID NO: 97 - RNA, 24 nt, Bos taurus
  source 1..24: mol_type = other RNA
gtcccccaggtgtgattctgattt

SEQ ID NO: 98 - RNA, 21 nt, Bos taurus
  source 1..21: mol_type = other RNA
taatgcccctaaaaatcctta

SEQ ID NO: 99 - RNA, 22 nt, Bos taurus
  source 1..22: mol_type = other RNA
taatgcccctaaaaatccttat

SEQ ID NO: 100 - RNA, 23 nt, Bos taurus
  source 1..23: mol_type = other RNA
cgcgacgagcccctcgcacaaac

SEQ ID NO: 101 - RNA, 23 nt, Bos taurus
  source 1..23: mol_type = other RNA
aggttacccgagcaactttgcat

SEQ ID NO: 102 - RNA, 22 nt, Bos taurus
  source 1..22: mol_type = other RNA
atgacacgatcactcccgttga

SEQ ID NO: 103 - RNA, 17 nt, Bos taurus
  source 1..17: mol_type = other RNA
accccactcctggtacc

SEQ ID NO: 104 - RNA, 17 nt, Bos taurus
  source 1..17: mol_type = other RNA
accccactcctggtacc

SEQ ID NO: 105 - RNA, 22 nt, Bos taurus
  source 1..22: mol_type = other RNA
tggcagtgtattgttagctggt

SEQ ID NO: 106 - RNA, 23 nt, Bos taurus
  source 1..23: mol_type = other RNA
agaactcttgcagtcttagatgt

SEQ ID NO: 107 - RNA, 22 nt, Bos taurus
  source 1..22: mol_type = other RNA
tcaggctcagtcccctcccgat

SEQ ID NO: 108 - RNA, 21 nt, Bos taurus
  source 1..21: mol_type = other RNA
agtggttatccctgctgtgtt

SEQ ID NO: 109 - RNA, 21 nt, Bos taurus
  source 1..21: mol_type = other RNA
gaggttatccgtgttgtcttc

SEQ ID NO: 110 - RNA, 23 nt, Bos taurus
  source 1..23: mol_type = other RNA
taatccttgctacctgggtgaga

SEQ ID NO: 111 - RNA, 19 nt, Bos taurus
  source 1..19: mol_type = other RNA
tgcacctgggcaaggattc

SEQ ID NO: 112 - RNA, 21 nt, Bos taurus
  source 1..21: mol_type = other RNA
aattcttgcccaggtgagagt

SEQ ID NO: 113 - RNA, 22 nt, Bos taurus
  source 1..22: mol_type = other RNA
accctcccacacccaaggcttg

SEQ ID NO: 114 - RNA, 21 nt, Bos taurus
  source 1..21: mol_type = other RNA
gatcttgttaaaaggcagatt

SEQ ID NO: 115 - RNA, 20 nt, Bos taurus
  source 1..20: mol_type = other RNA
cctttgagctgggaagggaa

SEQ ID NO: 116 - RNA, 20 nt, Bos taurus
  source 1..20: mol_type = other RNA
gctgatagactgagtcaggg

SEQ ID NO: 117 - RNA, 19 nt, Bos taurus
  source 1..19: mol_type = other RNA
gccggaactccgggtcctg

SEQ ID NO: 118 - RNA, 21 nt, Bos taurus
  source 1..21: mol_type = other RNA
aatggcgccactagggttgtg

SEQ ID NO: 119 - RNA, 22 nt, Bos taurus
  source 1..22: mol_type = other RNA
tctggggtaactttgagcaggg

SEQ ID NO: 120 - RNA, 20 nt, Bos taurus
  source 1..20: mol_type = other RNA
ctggggaaagcaggagtgag

SEQ ID NO: 121 - RNA, 18 nt, Bos taurus
  source 1..18: mol_type = other RNA
agaggtaaaaaggcacag

SEQ ID NO: 122 - RNA, 20 nt, Bos taurus
  source 1..20: mol_type = other RNA
ctgtgcttgcggctcccgcg

SEQ ID NO: 123 - RNA, 23 nt, Bos taurus
  source 1..23: mol_type = other RNA
tattcatttatctcccagcctac

SEQ ID NO: 124 - RNA, 22 nt, Bos taurus
  source 1..22: mol_type = other RNA
tgtgggtgtgtgcatgtgcgtg

SEQ ID NO: 125 - RNA, 24 nt, Bos taurus
  source 1..24: mol_type = other RNA
ggtttcctcatattcattcaggag

SEQ ID NO: 126 - RNA, 21 nt, Bos taurus
  source 1..21: mol_type = other RNA
tccctgagtatatgtggtgaa

SEQ ID NO: 127 - RNA, 21 nt, Bos taurus
  source 1..21: mol_type = other RNA
acaggcacggccagtttgagc

SEQ ID NO: 128 - RNA, 29 nt, Bos taurus
  source 1..29: mol_type = other RNA
gtttgctgaagccagatgccatttctgag

SEQ ID NO: 129 - RNA, 20 nt, Bos taurus
  source 1..20: mol_type = other RNA
gtagaggagatggcgcaggg

SEQ ID NO: 130 - RNA, 22 nt, Bos taurus
  source 1..22: mol_type = other RNA
tccattacactaccctgcctct

SEQ ID NO: 131 - RNA, 22 nt, Bos taurus
  source 1..22: mol_type = other RNA
tattgcacttgtcccggcctgt

SEQ ID NO: 132 - RNA, 22 nt, Bos taurus
  source 1..22: mol_type = other RNA
tattgcacttgtcccggcctgt

SEQ ID NO: 133 - RNA, 22 nt, Bos taurus
  source 1..22: mol_type = other RNA
gtgcagctgatcacagactgtg

SEQ ID NO: 134 - RNA, 20 nt, Bos taurus
  source 1..20: mol_type = other RNA
aggcaacgggcttggcggag

SEQ ID NO: 135 - RNA, 20 nt, Bos taurus
  source 1..20: mol_type = other RNA
tgtagaacatgttttggaac

SEQ ID NO: 136 - RNA, 22 nt, Bos taurus
  source 1..22: mol_type = other RNA
agctcactgaacaatgagtgca

SEQ ID NO: 137 - RNA, 23 nt, Bos taurus
  source 1..23: mol_type = other RNA
aaaccctgaaggaacattttggt

SEQ ID NO: 138 - RNA, 20 nt, Bos taurus
  source 1..20: mol_type = other RNA
aatgttcattccaatttttc

SEQ ID NO: 139 - RNA, 20 nt, Bos taurus
  source 1..20: mol_type = other RNA
tgattttgttgttttgtatt

SEQ ID NO: 140 - RNA, 22 nt, Bos taurus
  source 1..22: mol_type = other RNA
ctgcccgagcgcatgtggctga

SEQ ID NO: 141 - RNA, 22 nt, Bos taurus
  source 1..22: mol_type = other RNA
accggggagggggccggggatc